(19) United States
(12) Patent Application Publication
(10) Pub. No.: US 2008/0109069 A1
(43) Pub. Date: May 8, 2008

(54) BLOOD PERFUSION GRAFT

(76) Inventors: James E. Coleman, Terenure (IE); Christy Cummins, Naas (IE)

Correspondence Address: NUTTER MCCLENNEN & FISH LLP, WORLD TRADE CENTER WEST, 155 SEAPORT BOULEVARD, BOSTON, MA, 02210-2604, US

(21) Appl. No.: 11/557,293

(22) Filed: Nov. 7, 2006

(51) Int. Cl. A61F 2/06

(52) U.S. Cl. 623/1.25; 623/1.36; 623/2.1

(57) ABSTRACT

Methods and devices are provided for applying retrograde perfusion of blood at various locations within the body. In certain exemplary embodiments, the methods and devices are particularly useful during open or translumenal surgical approaches to apply long-term retrograde perfusion of the myocardium, the neurosystem, or a periphery, such as the arm or leg, thereby treating various medical conditions, such as coronary artery disease, stroke, renal failure, etc.

### FIELD OF THE INVENTION

The present invention relates to methods and device for perfusing blood.

### BACKGROUND OF THE INVENTION

Coronary artery disease is the leading cause of mortality and morbidity in the western world. A significant number of patients have diffuse coronary artery disease, absent conduits after bypass surgeries, small second vessels, or co-morbidities that may preclude Percutaneous Coronary Intervention (PCI) or Coronary Artery Bypass Grafting (CABG). It has previously been reported that approximately 5-7% of patient's with symptomatic obstructive coronary artery disease with documented ischemia who undergo coronary angiography at tertiary referral centers are not candidates for PCI or CABG. It has been estimated that there is approximately 7 million citizens in the United States with angina pectoris, and an estimated 350,000 new cases occur each year. Of approximately 2 million cardiac catheterizations performed in the United States in 2005 and based on approximately 5% of patient's being ineligible for conventional revascularization, 100,000 to 150,000 patients per year may be eligible for new methods for revascularization. These so called “no-option” patients have frequently diffuse coronary disease without a discrete target for angioplasty, stenting or surgical bypass. Gene therapy and laser revascularization strategies to create new blood vessels in ischemic myocardium so far have not been convincingly successful. Myocardial tissue requires significant arterial inflow and is not obviously provided by these techniques.

Therefore, alternative routes and techniques to improve the myocardial perfusion in these patients appear warranted. In 1927, Wearns was able to reveal that upon blockage of the coronary veins, 90% of the venous blood will drained back into the heart by the Thebesian system. Thus, arterialization of the veins should be possible without risking detrimental congestion. As shown by several surgical series, arterialization of the coronary veins indeed has the potential of providing strong arterial inflow to a severely ischemic region and to improve the symptoms of patients with severe angina. For example, Park showed in 1975 in a paper titled “Direct Selective Myocardial Revascularisation By Internal Mammary Artery Coronary Vein Anastomosis” in the Journal of Thoracic Cardiovascular Surgery, that in six patients with diffuse left anterior descending (LAD) disease receiving a left internal mammary artery (LIMA) graft to the anterior interventricular vein (AIV), all patients remained symptom free of angina after one year. In 2000 the first case of Percutaneous In-situ Coronary Venous Arterialization (PICVA) was performed on a human. A further ten patients were treated in the PICVA European Safety Trial with mixed results. However, the author concluded that on further developments of the technology, the catheter based coronary bypass procedure could become a broad based interventional application.

Accordingly, there remains a need for improved methods and devices for improving myocardial perfusion, as well as methods and devices for perfusing blood to various other locations within the body.

### SUMMARY OF THE INVENTION

The present invention generally provides methods and devices for perfusing blood. In one exemplary embodiment, a blood perfusion device is provided and includes an implantable hollow flexible conduit configured to be implanted in a human heart. The conduit can include first and second ends and a plurality of perforations formed in a sidewall thereof and configured to decrease a pressure of fluid flowing through the conduit. The device can also include at least one expandable anchor formed on the conduit and adapted to expand to engage tissue to anchor at least a portion of the conduit to the tissue. The expandable anchor(s) can have a plurality of openings formed therethrough and in communication with the hollow conduit such that blood can flow through the plurality of openings and through the hollow conduit.

The conduit can have a variety of configurations, but in one exemplary embodiment the conduit is formed from a metal or a polymer. The quantity and size of the perforations in the conduit can also vary, but in an exemplary embodiment the quantity and size of perforations is configured to maintain a maximum pressure within the conduit that corresponds to a maximum pressure obtained within the coronary sinus of a human heart. The perforations can be formed at any location along the conduit, such as along a substantial portion of a length of the conduit.

The expandable anchor(s) can also have a variety of configurations, but in one the expandable anchor is in the form of a first expandable anchor formed on the first end of the conduit, and a second expandable anchor formed on the second end of the conduit. The first expandable anchor can include first and second expandable portions configured to engage tissue therebetween, and the second expandable anchor can be formed from a mesh material to allow blood to flow freely therethrough.

In another embodiment, the conduit can include first and second conduit portions that are matable to one another. The first conduit portion can have the first expandable anchor formed on a terminal end thereof, and the second conduit portion can have the second expandable anchor formed on the terminal end thereof. The device can also include other features, such as a one-way valve disposed within at least one of the conduit and the at least one expandable anchor for controlling a direction of blood flow through the device, and/or a cardiac pacing wire disposed through the conduit and at least one of the openings in the at least one expandable anchor.

In another exemplary embodiment, a bypass device is provided and includes a flexible elongate conduit configured to be implanted in a human heart. The conduit can include a lumen extending therethrough and configured to direct blood from a left ventricle, across an interventricular septum, through a right ventricle, and into a coronary sinus of the heart. The conduit can also include a plurality of perforations having a size and a quantity configured to maintain a maximum pressure within the conduit that corresponds to a maximum pressure obtained within the coronary sinus of a human heart, and at least one expandable anchor configured to engage tissue and anchor at least a portion of the conduit to the tissue. The expandable anchor preferably has a plurality of openings configured to allow blood to flow therethrough. The device can have a variety of configurations and features, such as those previously described above.

Exemplary methods are also provided for treating various medical conditions. In one embodiment, a method for treating heart disease is provided and includes anchoring a first end of a bypass device within an interventricular septum formed between left and right ventricles of a heart, and positioning a second end of the bypass device within a coronary ostium of the heart. The bypass device can have a hollow conduit extending from the first end of the device, through the right ventricle, across a tricuspid valve, through the right atrium, into the coronary sinus, to the second end of the device in the coronary ostium such that blood flows from the left ventricle, into the first end, through the conduit, and out the second end into the coronary sinus. In one exemplary embodiment, the conduit can include a plurality of perforations formed therein that decrease a pressure of blood flowing through the conduit. Positioning the second end of the bypass device within a coronary ostium of the heart can further include anchoring the second end of the bypass device within the coronary ostium. Various anchoring techniques can be used including, for example, removing a sheath disposed around an expandable anchor located on the first end to allow the expandable anchor to expand to engage tissue, advancing the expandable anchor from within the conduit to allow the expandable anchor to expand to engage tissue, or inflating a balloon disposed within an expandable anchor located on the first end to expand the expandable anchor such that the expandable anchor engages tissue. The method can also include positioning a cardiac pacing wire through the bypass device and into tissue in the heart.

In another embodiment, anchoring the first end of the bypass device can include advancing a guidewire through the right atrium and through a puncture formed in the interventricular septum, advancing the conduit of the device over the guidewire to position the first end of the bypass device within the left ventricle, and expanding an expandable anchor located on the first end of the bypass device to cause the expandable anchor to engage the interventricular septum, thereby anchoring the first end within the interventricular septum.

In one embodiment, positioning the second end of the bypass device can include inserting a second guidewire through the aorta, into the left ventricle, into the first end and through the conduit of the bypass device, and into the coronary sinus to position a leading end of the second guidewire within the coronary ostium, and advancing the second end of the bypass device along the guidewire such that the second end of the bypass device is advanced into the coronary ostium. The second end of the bypass device can be advanced along the guidewire by, for example, advancing a catheter over the second guidewire to position an expandable member on the catheter within and adjacent to the second end of the bypass device, expanding the expandable member on the catheter to engage the second end of the bypass pass, and advancing the catheter along the guidewire to advance the second end along the guidewire and thereby position the second end in the coronary ostium. In another embodiment, the method can include expanding an expandable anchor located on the second end of the bypass device to cause the expandable anchor to engage and anchor the second end of the bypass device within the coronary ostium. The expandable anchor can be expanded, for example, by advancing a pusher over the second guidewire and through conduit to push an expandable anchor contained within the second end out of the second end whereby the expandable anchor expands to engage tissue.

In yet another embodiment, the conduit can include first and second conduit portions slidably matable to one another, and positioning the first and second ends of the bypass device can include advancing a first guidewire through the right atrium and through a puncture formed in the interventricular septum, advancing the first conduit portion of the bypass device over the first guidewire to position the first end of the bypass device within the left ventricle, and expanding at least one expandable anchor located on the first end of the first conduit portion to cause the expandable anchor to engage the interventricular septum, thereby anchoring the first end within the interventricular septum. The method can further include advancing the second conduit portion over the first guidewire to slidably mate the second conduit portion to the first conduit portion, removing the first guidewire, inserting a second guidewire through the aorta, into the left ventricle, into the first end and through the conduit of the bypass device, and into the coronary sinus to position a leading end of the second guidewire within the coronary ostium, and advancing the second end of the bypass device along the second guidewire such that the second end of the bypass device is advanced into the coronary ostium. The expandable anchor(s) located on the first end of the first conduit portion can be expanded by, for example, withdrawing a sheath disposed over the first end of the first conduit portion to allow first and second expandable portions located on the first end of the first conduit portion to expand and engage the interventricular septum therebetween. In another embodiment, the second end of the bypass graft can be advanced along the second guidewire by advancing a catheter over the second guidewire to position an expandable member on the catheter within and adjacent to the second end of the bypass device, expanding the expandable member on the catheter to engage the second end of the bypass pass, and advancing the catheter along the second guidewire to advance the second end along the guidewire and thereby position the second end in the coronary ostium. In other aspects, the method can include expanding an expandable anchor located on the second end of the bypass device to cause the expandable anchor to engage and anchor the second end of the bypass device within the coronary ostium. The expandable anchor located on the second end can be expanded by, for example, advancing a pusher over the second guidewire and through the conduit to push the expandable anchor contained within the second end out of the second end whereby the expandable anchor expands to engage tissue.

In yet another embodiment, a method for treating heart disease is provided and includes positioning a hollow elongate conduit within a heart to re-direct blood flow through the conduit from a left ventricle, through an interventricular septum, through the right ventricle, through the right atrium, into the coronary sinus, and into the coronary ostium. The conduit can include a plurality of perforations formed therein and configured to maintain a maximum pressure within the conduit that corresponds to a maximum pressure obtained within the coronary sinus of a human heart. In an exemplary embodiment, the hollow elongate conduit can include a first end that is anchored within the interventricular septum, and a second end that is positioned in the coronary ostium. The second end can be configured to allow blood to flow therethrough and to at least partially occlude the coronary ostium. The method can also include removing the device after an extended period of use.

### BRIEF DESCRIPTION OF THE DRAWINGS

The invention will be more fully understood from the following detailed description taken in conjunction with the accompanying drawings, in which:

FIG. 1A is a perspective view of one embodiment of a bypass graft having a conduit with expandable anchors located on second ends thereof;

FIG. 1B is an enlarged view of one of the expandable anchors of the device of FIG. 1A;

FIG. 1C is an enlarged view of the other expandable anchor of the device of FIG. 1A;

FIG. 1D is a cross-sectional view of another embodiment of a distal end portion of the expandable anchor of FIG. 1C;

FIG. 1E is a partially transparent view of the conduit of FIG. 1A;

FIG. 1F is a perspective view of another embodiment of a bypass graft;

FIG. 1G is a side view of the device of FIG. 1F shown in a curved position to prevent kinking;

FIG. 1H is a side view of a portion of the device of FIG. 1F implanted within a body lumen;

FIG. 1I is a side view of one embodiment of a support device for use with the various grafts disclosed herein;

FIG. 2 is a disassembled perspective view of another embodiment of a bypass graft having a conduit with first and second conduit portions, each having an expandable anchor located on a terminal end thereof;

FIG. 3 is a partially disassembled perspective view of another embodiment of a bypass graft having a conduit with first and second conduit portions, each having an expandable anchor located on opposed ends thereof;

FIG. 4A is a perspective view of yet another embodiment of a bypass graft having a conduit formed from two portions, each having an expandable anchor located on opposed ends thereof;

FIG. 4B is an enlarged view of a portion of the device of FIG. 4A;

FIG. 5A is a perspective view of another embodiment of an expandable anchor having first and second wing portions;

FIG. 5B is a side view of the device of FIG. 5A showing the wings portions deployed and engaging tissue therebetween;

FIG. 6A is a perspective view of the device of FIG. 5A, showing a second expandable anchor formed on an opposite end thereof;

FIG. 6B is a perspective view of the expandable anchor of FIG. 6A, showing the anchor retracted within the conduit;

FIG. 6C is a perspective vie of the expandable anchor of FIG. 6B, showing the anchor deployed;

FIG. 7A is a perspective view of another embodiment of an expandable anchor, showing the anchor in a deployed configuration;

FIG. 7B is a perspective view of the expandable anchor of FIG. 7A, showing the anchor retracted prior to deployment;

FIG. 8A is a perspective view of yet another embodiment of an expandable anchor having wires couples to a deployment ring, showing the deployment ring in the retracted position;

FIG. 8B is a perspective view of the device of FIG. 8A, showing the deployment ring in the extended position to expand the wires;

FIG. 9A is a perspective view of an expandable anchor having a coiled configuration, showing the anchor in the compressed position;

FIG. 9B is a perspective view of the anchor of FIG. 9A, showing the anchor in the expanded position;

FIG. 10A is a perspective view of another embodiment of an expandable anchor having wires disposed within circumferentially-oriented slots formed in a conduit, showing the anchor in the retracted position;

FIG. 10B is a perspective view of the anchor of FIG. 10A, showing the anchor in the expanded position;

FIG. 11A is a perspective view of another embodiment of an expandable anchor formed from several loop-shaped wires, showing the anchor in a compressed position;

FIG. 11B is a perspective view of the anchor of FIG. 11A, showing the anchor in the expanded position;

FIG. 12A is a perspective view of another embodiment of an expandable anchor formed from several hook-shaped wire strips, showing the anchor in a retracted position;

FIG. 12B is a perspective view of the anchor of FIG. 12A, showing the anchor in the expanded position;

FIG. 13 is a perspective view of yet another embodiment of an expandable anchor formed from several hook-shaped wires, showing the anchor in an expanded position;

FIG. 14 is a perspective view of yet another embodiment of a bypass graft having a conduit and expandable anchors that are formed from a coiled wire;

FIG. 15 is a cross-sectional view of a human heart, showing a graft positioned to perfuse blood from the left ventricle to the coronary sinus;

FIG. 16 is a cross-sectional view of a human heart, showing another embodiment of a graft positioned to perfuse blood from the left ventricle to the coronary sinus and having mating ends that are positioned within the right ventricle;

FIG. 17 is a cross-sectional view of a human heart, showing the graft of FIG. 16 with the mating ends located at the opening of the coronary sinus;

FIG. 18 is a cross-sectional view of a human heart, showing another embodiment of a graft positioned to perfuse blood from the left ventricle to the coronary sinus;

FIG. 18A is a side view of another embodiment of a technique for anchoring a graft within the mitral valve;

FIGS. 19A-19H illustrate one exemplary translumenal method for implanting a graft within a heart to perfuse blood from the left ventricle to the coronary sinus;

FIGS. 20A-20H illustrate another exemplary translumenal method for implanting a graft within a heart to perfuse blood from the left ventricle to the coronary sinus;

FIGS. 21A-21H illustrate yet another translumenal method for implanting a graft within a heart to perfuse blood from the left ventricle to the coronary sinus;

FIGS. 22A-22H illustrate another translumenal method for implanting a graft within a heart to perfuse blood from the left ventricle to the coronary sinus;

FIGS. 23A-23H illustrate another embodiment of a translumenal method for implanting a graft within a heart to perfuse blood from the left ventricle to the coronary sinus;

FIGS. 24A-24F illustrate an exemplary embodiment of a trans-septal method for implanting a graft within a heart to perfuse blood from the left ventricle to the coronary sinus;

FIGS. 25A-25M illustrate yet another exemplary translumenal method for implanting a graft within a heart to perfuse blood from the left ventricle to the coronary sinus;

FIGS. 26A-26F illustrate an exemplary method for implanting a graft using conventional surgical procedures to position the graft within a heart to perfuse blood from the left ventricle to the coronary sinus;

FIGS. 27A-27G illustrate an exemplary method for implanting a graft to perfuse blood from the left ventricle to the coronary vein;

FIGS. 28A-28D illustrate an exemplary method for implanting a graft through a support device anchored within the interventricular septum;

FIG. 29 illustrates a method for implanting a graft to perfuse blood from the left ventricle to a region of the brain;

FIG. 30 illustrates another exemplary method for implanting a graft to perfuse blood from the left ventricle to a region of the brain; and

FIG. 31 illustrates an exemplary method for implanting a graft to perfuse blood from the left ventricle to a periphery.

### DETAILED DESCRIPTION OF THE INVENTION

Certain exemplary embodiments will now be described to provide an overall understanding of the principles of the structure, function, manufacture, and use of the devices and methods disclosed herein. One or more examples of these embodiments are illustrated in the accompanying drawings. Those of ordinary skill in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The present invention generally provides methods and devices for applying retrograde perfusion of blood at various locations within the body. In certain exemplary embodiments, the methods and devices are particularly useful during open or translumenal surgical approaches to apply long-term retrograde perfusion of the myocardium, the neurosystem, or a periphery, such as the arm or leg, thereby treating various medical conditions, such as coronary artery disease, stroke, renal failure, etc. A person skilled in the art will appreciate that the various methods and devices disclosed herein can be used to treat a variety of medical conditions.

In one exemplary embodiment, a graft is provided and is adapted to be implanted translumenally in a patient's body to perfuse blood in a retrograde manner for treating various medical conditions. While the particular configuration of the graft can vary depending on the intended use, in general the graft can include a conduit having an elongate configuration with first and second ends and an inner lumen extending therethrough between the first and second ends. The conduit can be formed from a single elongate member, or it can be formed from two or more elongate members that are fixedly or more preferably removably matable to one another. The sidewalls of conduit can include one or more perforations formed therein at various locations along the length of the conduit for allowing blood flow therethrough. The particular quantity, size, and location of the perforations can vary depending on the intended use of the device, exemplary embodiments of which will be discussed in more detail below. The conduit can also be formed from a variety of materials, but in an exemplary embodiment the conduit is flexible to facilitate translumenal introduction. The type of material can be varied to obtain the desired flexibility and/or the perforations can be adapted to facilitate flexibility of all or various portions of the conduit. In one exemplary embodiment, the conduit can be formed from plastic, or a metal such as stainless steel, nitinol, or titanium. Various techniques can also be used to form the conduit, including braiding, weaving, laser cutting, wire coiling, or other techniques known in the art for forming a conduit.

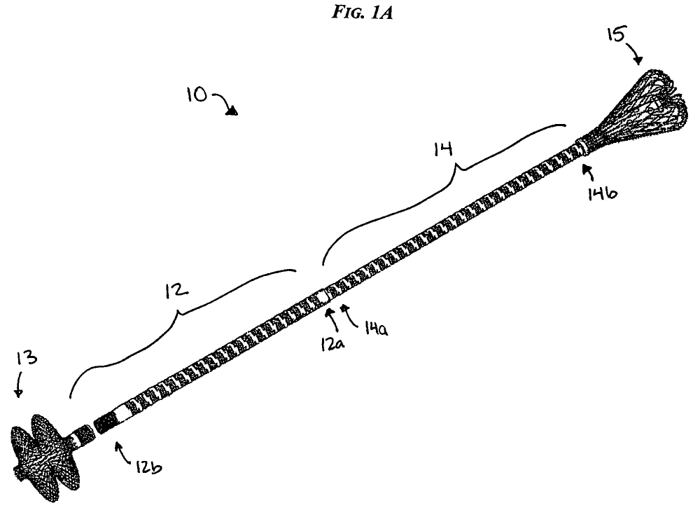

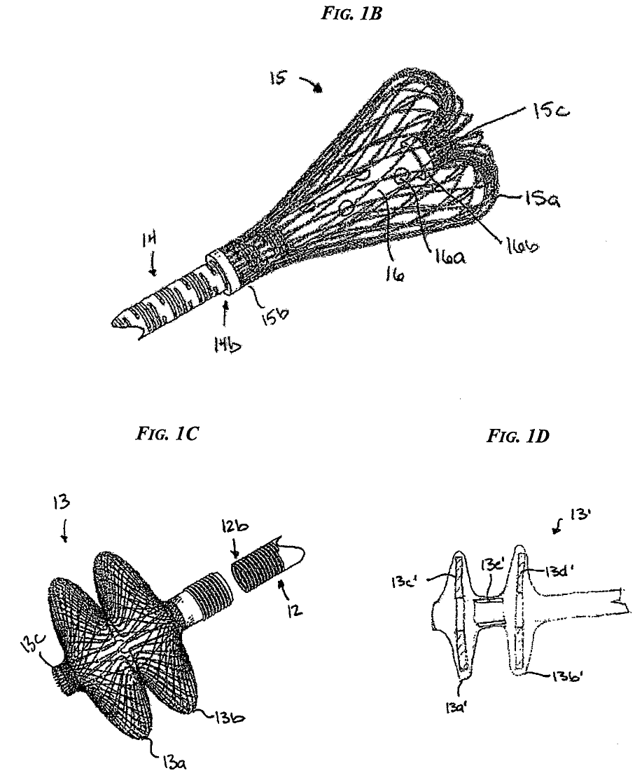

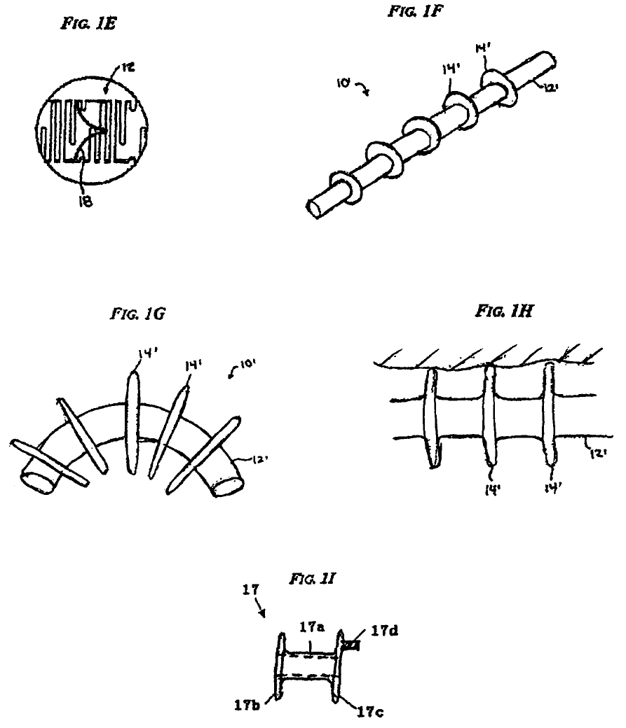

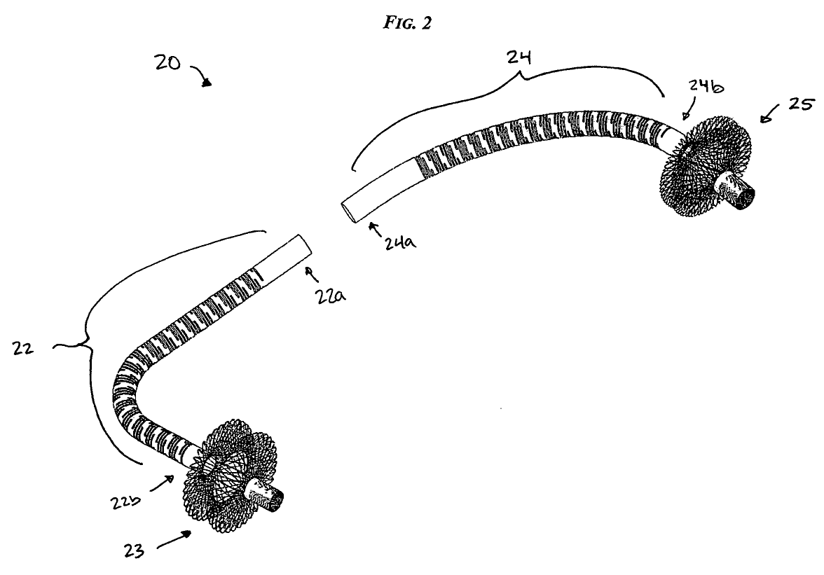

In an exemplary embodiment, the conduit includes at least one expandable anchor formed thereon. The anchor(s) can be located at the first and/or second ends, or at a location between the first and second ends. The particular location of the expandable anchor(s), as well as the quantity of expandable anchors, can vary depending on the intended use of the graft. In use, the expandable anchor is preferably adapted to be introduced into a body lumen in a compressed (i.e., a non-expanded configuration), and is adapted to expand to engage the body lumen and thereby anchor the conduit within the body lumen. FIGS. 1A-14 illustrate various exemplary embodiments of grafts formed from conduits having expandable anchors located thereon. A person skilled in the art will appreciate that a graft can be configured having any one of the anchors shown in FIGS. 1A-14 formed along any portion of the conduit.

FIG. 1A illustrates one exemplary embodiment of a graft 10 having a hollow conduit that is formed from two portions: a first conduit portion 12 and a second conduit portion 14. Each conduit portion 12, 14 includes a mating end 12a, 14a and a terminal end 12b, 14b. The mating ends 12a, 14a are removably mated to one another to form a generally elongate conduit. Various mating techniques can be used including, for example, an interference fit, a threaded engagement, a snap-fit, etc. As further shown in FIG. 1A, the graft 10 further includes a first expandable anchor 13 formed on the terminal end 12b of the first conduit portion 12, and a second expandable anchor 14 formed on the terminal end 14b of the second conduit portion 14. The expandable anchors 13, 15 can be fixedly attached to the conduit portions 12, 14, or they can be removable coupled to the conduit portions 12, 14 as shown using, for example, threads, a snap-fit, an interference fit, or other engagement techniques. Each expandable anchor 13, 15 can vary in shape and size, but in an exemplary embodiment each anchor 13, 15 has a generally elongate tubular shape in the compressed position to allow it to be introduced into a body lumen, and an enlarged three-dimensional shape that is adapted to fit within or engage a particular body region when in the expanded position. The anchors 13, 15 can also have an inner lumen extending partially or fully therethrough and in communication with the inner lumen of the hollow conduit to allow blood to flow from the conduit through the anchors 13, 15. Each anchor 13, 15 can also be formed from a porous or perforated material that allows blood to flow through the sidewalls of the anchors 13, 15. The quantity, shape, and size of the perforations can be configured to control the amount of blood flow therethrough as may be desired. In the illustrated embodiment, the first expandable anchor 13 on the first conduit portion 12 is adapted to be anchored across the interventricular septum, and the second expandable anchor 15 on the second conduit portion 14 is adapted to be anchored within the coronary sinus.

FIG. 1B illustrates the second expandable anchor 15 in more detail, and as shown the second expandable anchor 15 has a generally elongate or oblong configuration in the expanded position. The anchor 15 includes a hollow tubular body 16 extending therethrough and having an inner lumen in fluid communication with the inner lumen of the conduit. The tubular body 16 can include a number of perforations 16a formed therein, as shown, to allow blood flow therethrough. In other embodiments, the tubular body 16 can be integral with the second conduit portion 14 such that the terminal end 14b of the second conduit portion 14 extends through the anchor 15. The anchor 15 also includes a series of interwoven wires 15a that have a mating end 15b coupled to the terminal end 14b of the second conduit portion 14, and a terminal end 15c coupled to the terminal end 16b of the tubular body 16.

In use, one or more portions of the anchor 15 can be slidably movable relative to the second conduit portion 14 to allow the anchor 15 to move between the compressed and expanded configurations. For example, the tubular body 16 can slide relative to the conduit portion 14 such that extension of the tubular body 16 from the conduit portion 14 will pull the wires 15a and thereby compress the anchor 15, and retraction of the tubular body 16 at least partially into the conduit portion 14 will allow the wires 15a to expand radially outward, as shown in FIG. 1B. Alternatively, full retraction of the tubular body 16 into the conduit portion 14 can pull the wires 15a into the conduit portion 14, thereby compressing the anchor 15. In another embodiment, the anchor 15 can be formed from an inflatable balloon or it can be self-inflating. By way of non-limiting example, the wires 15a can be formed from a shape memory material, such as nitinol. In use, the expandable anchor 15 can be biased to the expanded position, and it can be compressed by, for example, retracting the anchor 15 into a sleeve or the conduit portion 14. Advancement or removal of the anchor 15 from the sleeve or conduit portion 14 will allow the anchor 15 to return to the expanded position, whereby the anchor 15 is effective to engage tissue. Exemplary methods for use will be discussed in more detail below.

FIG. 1C illustrates the first expandable anchor 13 in more detail, and as shown the first expandable anchor 13 includes first and second expandable portions 13a, 13b that are configured to engage tissue therebetween in the expanded position. Each expandable portion 13a, 13b can vary in shape and size, but they preferably have a diameter or width that is sufficient to allow a tissue surface to be engaged between the two portions 13a, 13b. The expandable portions 13a, 13b can also be separate from one another with a connector extending therebetween or, as shown in FIG. 1C, the first and second expandable portions 13a, 13b can be formed from a single expandable body having a mid-portion with a diameter that is sufficiently smaller than a diameter of the first and second expandable portions 13a, 13b. In an exemplary embodiment, the mid-portion is configured to be positioned within an interventricular septum to allow blood to flow therethrough.

In another embodiment, shown in FIG. 1D, each portion 13a′, 13b′ of the first expandable anchor 13′ can include a disc 13c′, 13d′ or other structure disposed therein. The discs 13c′, 13d′ can be formed from various materials, including various biocompatible materials such as a polyester fiber (e.g., Dacron®). Each disc 13c′, 13d′ can have a center hole extending there through for allowing fluid to pass from the conduit through the anchor 13′. The outer sections of the discs 13c′, 13d′ can, however be configured to prevent the passage of blood through the expanded sections 13a′, 13b′ of the anchor 13′. The anchor 13′ can also include a reinforcing member 13e′ positioned within the non-expanded section of the anchor 13′ that is located between the expanded portions 13a′, 13b′. The reinforcing member 13e′ can be a tubing, additional wire strands, a metal insert, or other structure to prevent collapse of the non-expanded section, as well as the tissue surrounding the non-expanded section.

In another embodiment, rather than having an internal reinforcing member, the various grafts disclosed herein can be used in combination with a separate support device. For example, the pressure resulting from a beating heart can cause an opening in the interventricular septum to close, thus a support device can optionally be positioned within the opening in the septum to prevent the opening from closing. A graft can be passed through the support device and anchored to the septum using the various anchoring structures disclosed herein. By way of non-limiting example, FIG. 1I illustrates one exemplary embodiment of a support device 17 that includes a hollow tubular body 17a having first and second expandable anchors 17b, 17c formed on opposed ends thereof. The anchors 17b, 17c can have various configurations, but in the illustrated embodiment each anchor 17b, 17c is in the form of a flexible substantially circular-shaped wing such that the anchors 17b, 17c are configured to engage tissue therebetween. In use, the anchors 17b, 17c can be folded or deformed to allow the support member 17 to be positioned within, for example, a delivery catheter. Retraction of the delivery catheter from around the anchors 17b, 17c will allow the anchors 17b, 17c to return to the expanded configuration, shown in FIG. 1I, thus allowing the anchors 17b, 17c to engage tissue. As further shown in FIG. 1I, the support member 17 can also optionally include an attachment 17d configured to mate to a delivery device, such as a delivery wire. The attachment 17d can have a variety of configurations, and it can be located at various locations along the anchor. In the illustrated embodiment, the attachment 17d is in the form of a small tubing or a protruding pin that is positioned on an external facing surface of one of the anchors. The tubing or pin is configured to receive and mate to a terminal end of a delivery device therein. Various mating techniques can be used including, for example, threads, a press-fit, or other techniques known in the art.

Referring back to FIG. 1C, an extension portion 13c is also shown extending from a terminal end of the first expandable portion 13a. The extension portion 13c allows a portion of the anchor 13 to extend beyond a tissue surface engaged between the two portions 13a, 13b of the anchor 13. As further shown in FIG. 1C, the anchor 13 can be removably mated to the terminal end 12b of the conduit portion 12. FIG. 1C illustrates threads formed within the terminal end 12b of the first conduit 12, and corresponding threads formed on a mating end of the anchor 13. The first expandable anchor 13 can also be formed from a variety of materials, including those previously discussed with respect to the second expandable anchor 15, to allow the anchor 13 to move between the compressed and expanded positions.

As shown in FIG. 1E, the conduit can also optionally include a one-way valve disposed therein for controlling a direction of blood flow therethrough. FIG. 1E illustrates the first conduit portion 12 having a one-way valve 18 disposed within the inner lumen thereof for allowing fluid flow therethrough in only one direction. The use of a one-way valve can also prevent suction of blood from the conduit into the left ventricle during cardiac diastole. A person skilled in the art will appreciate that the location and quantity of one-way valves can vary depending on the intended use, and various one-way valves known in the art can be used.

FIGS. 1F-1H illustrate another embodiment for anchoring a graft within a body lumen. As shown, the graft 10′ includes a conduit portion 12′ having several disc elements 14′ disposed around an external surface thereof and spaced apart at intervals along a longitudinal axis of the conduit 12′. The disc elements 14′ can be separate members that are fixedly attached to an external surface of the conduit 12′, or they can be formed by compressing portions of the conduit 12′ together to cause the compressed section to collapse and increase in diameter, as shown in more detail in FIG. 1H. During manufacturing, the conduit 12′ can optionally be heat treated to retain the collapsed configuration. The conduit 12′ and discs 14′ can also optionally be formed from a flexible material to allow the conduit 12′ to be extended in length if required. For example, a tensile force can be applied to the conduit 12′ to reduce a diameter of discs 14′ and to increase a length of the conduit 12′. In use, as shown in FIG. 1H, the disc members 14′ can prevent the conduit 12′ from contacting tissue or other structures which might cause occlusion of any perforations in the wall of the conduit 12′. The disc 14′ can also prevent the collapse or kinking of the conduit 12′ when formed into a curved configuration, as shown in FIG. 1G. In this position the discs 14′ are positioned adjacent to or in contact with each other on the inner side of the curve preventing further collapse of the conduit 12′.

FIG. 2 illustrates another embodiment of a graft 20 having a conduit that is formed from first and second conduit portions 22, 24 that are removably matable to one another at a mating end 22a, 24a thereof. Each conduit portions includes an expandable anchor 23, 25 formed on a terminal end 22b, 24b thereof. In this embodiment, the conduit portions 22, 24 are preformed to have a curved shape to better facilitate placement within the heart. In use, a delivery device, such as a guidewire, can be used to deform the conduit portions 22, 24 to facilitate insertion through a body lumen. Subsequent removal of the delivery device will allow the conduit portions 22, 24 to return to their deformed configuration, thereby taking on the desired shape and preferably facilitating positioning of the graft 20 in a desired location.

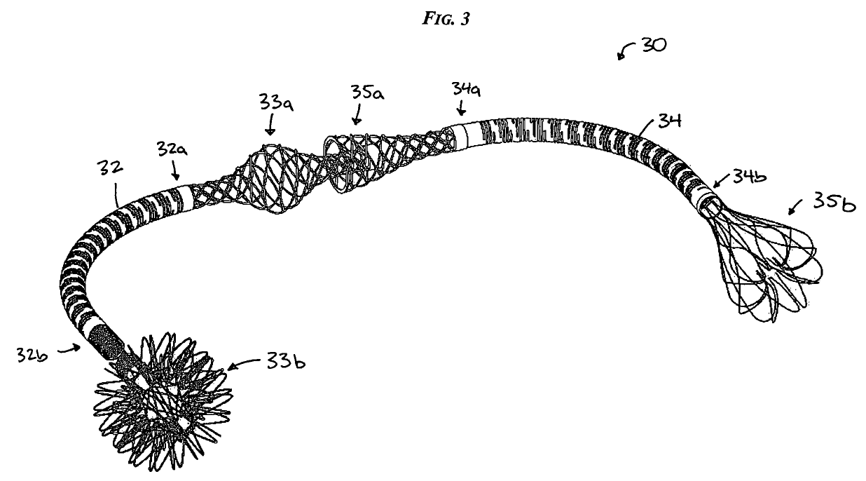

In another embodiment, shown in FIG. 3, the mating ends of the first and second portions of the conduit can also include expandable anchors. FIG. 3 illustrates a graft 30 having a conduit formed from first and second conduit portions 32, 34, each of which includes a mating end 32a, 34a and a terminal end 32b, 34b. As shown, the mating end 34a of the second conduit portion 34 includes an expandable anchor 35a formed thereon and having a generally conical shape in the expanded configuration, and the mating end 32a of the first conduit portion 32 includes an expandable anchor 33a formed thereon and having a generally bulbous oblong shape that is configured to be received within the expandable anchor 35a on the second conduit portion 34. With the anchor 35a on the second conduit portion 34 in the expanded configuration, the anchor 33a on the first conduit portion 33 can be inserted therein and expanded to engage the anchor 35a on the second conduit portion 34, thereby mating the two portions 32, 34 to one another. This expandable mid-portion of the conduit can be positioned at various locations within the body, and it can serve various purposes. For example, the expandable mid-portion can merely function to connect the first and second conduit portions 32, 43, or it can act as a valve depending on the particular configuration of the mid-portion to control blood flow therethrough thus increasing or decreasing the pressure within the conduit. The mid-portion can also function as base for mating other components to the graft 30. For example, a control wire or other device for delivering and/or retrieving the mating ends 32a, 34a of the first and second conduit portion 32, 34 can be mated to the anchors 33a, 35a. In another embodiment, the mid-portion can function to allow movement between the first and second conduit portions 32, 34. For example, the mid-portion can form a ball-and-socket joint that allows rotation between the two conduit portions 32, 34 during, for example, the cardiac cycle. A person skilled in the art will appreciate that the mid-portion can have a variety of configurations and the particular configuration can be adapted based on the intended use.

FIG. 3 also illustrates alternative embodiments of expandable anchors 33b, 35b formed on the terminal end 32b, 34b of each conduit portion 32, 34. As shown, the expandable anchor 33b formed on the terminal end 32b of the first conduit portion 32 is formed from a series of wires, each having a generally triangular shape and extending laterally outward to form a generally spherical anchor. The expandable anchor 35b formed on the terminal end 34b of the second conduit portion 34 is also formed from a series of wires, however the wires having a first end that is mated to the terminal end 34b of the second conduit portion 34, and a second end that is hook- or U-shaped to form a bulbous region.

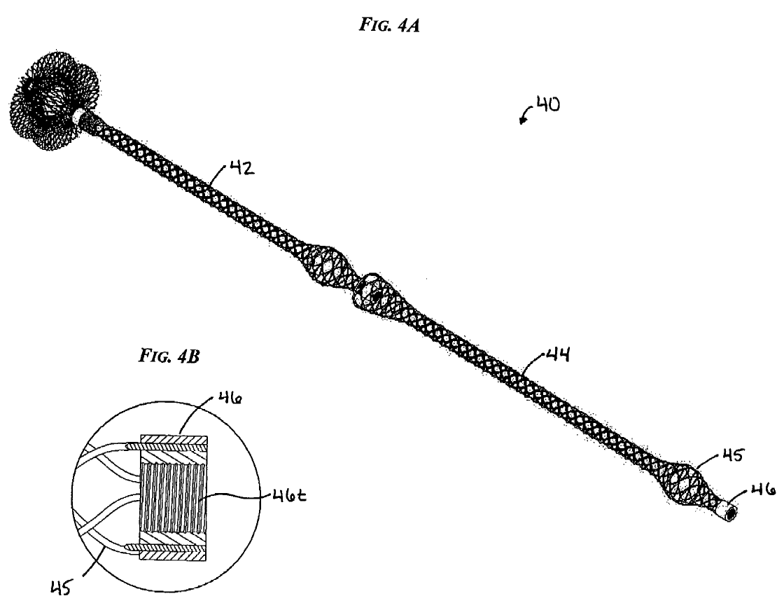

In another embodiment, the expandable anchors can include features to facilitate mating to an actuator, a steering mechanism, or other devices. FIG. 4A illustrates a graft 40 having first and second conduit portions 42, 44 that are removably matable to one another. The second conduit portion 44 includes an expandable anchor 45 formed on the terminal end thereof. The expandable anchor 45 has a cylindrical fitting 46 mated thereto. The fitting 46 is shown in more detail in FIG. 4B, and as shown the wires that form the anchor 45 are gathered and attached to the fitting 46. The fitting is particularly useful as it can allow other devices, such as a steering wire, to be mated thereto to facilitate steering of the conduit through a body lumen during use of the graft 40. The fitting 46 can also be used to allow an actuator to be attached thereto for moving the expandable anchor 45 between the compressed and expanded positions. As shown in FIG. 4B, an internal bore of the fitting 46 includes threads 46t formed therein. A threaded shaft can be disposed through the conduit and it can mate to the threads 46t of the fitting 46 to move the fitting 46 along a longitudinal axis of the graft 40. Movement of the fitting 46 in a first direction can stretch the anchor 45 to compress it, and movement of the fitting 46 in an opposite direction can return the anchor 45 to the expanded position. Alternatively, the threaded shaft can maintain the anchor 45 in the compressed configuration, and unthreading the shaft from the fitting 46 will allow the anchor 45 to axially compress and thereby radially expand to engage tissue.

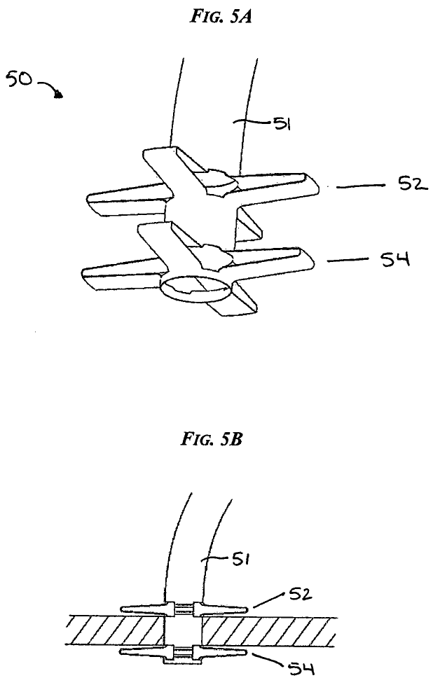

FIGS. 5A and 5B illustrate another embodiment of a graft 50 having a conduit 51 with an expandable anchor formed thereon. In this embodiment, the expandable anchor includes first and second expandable portions 52, 54 that are adapted to engage tissue therebetween. The first and second expandable portions 52, 54 can be formed by cutting two sets of longitudinally-extending slots in the conduit 51 such that the sidewalls of the conduit 51 extending between the slots can deform radially outward upon compression of the conduit 51. When expanded, the sidewalls form parallel sets of wings that can engage tissue therebetween, as shown in FIG. 5B. Alternatively, the anchor can optionally be biased to the expanded position shown in FIGS. 5A and 5B, and an actuator or a sheath can be used to compress the anchor into a compressed configuration for delivery.

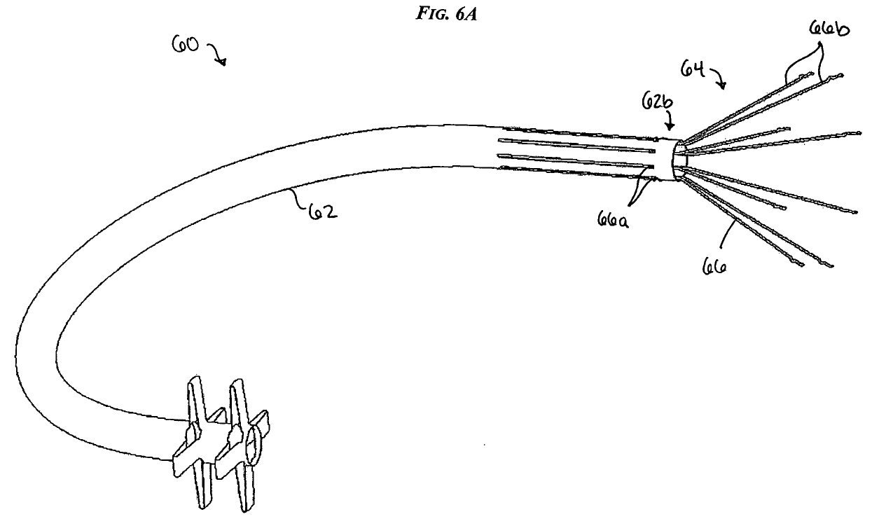

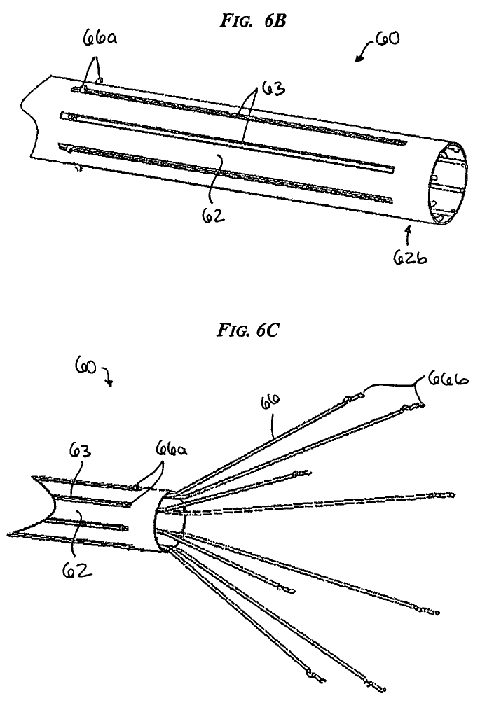

FIG. 6A illustrates another embodiment of a graft 60 having a conduit 62 with an expandable anchor 64 formed on a terminal end 62b thereof. In this embodiment, the expandable anchor 64 is formed from several wires 66 that are spaced around a perimeter of the conduit 62 and that extend radially outward from the terminal end 62b of the conduit 62. In particular, each wire 66 includes a mating end 66a that is mated to the conduit 62, and a terminal end 66b that extends radially outward from the terminal end 62b of the conduit. The mating ends 66a of the wires 66 are slidably coupled to the conduit 62 to allow movement between a compressed position, in which the wires 66 are retained within the conduit 62, and the extended position as shown in FIG. 6A. In the extended position, the wires 66 can be biased radially outward to allow the expandable member to anchor the conduit 62 within a body lumen. While various techniques can be used to slidably couple the wires 66 to the conduit 62, in one exemplary embodiment each wire 66 includes a curved element formed on the mating end 66a thereof and disposed within a cut-out or longitudinally extending slot 63 formed in the conduit 62, as shown in more detail in FIG. 6B. The curved mating end 66a can be configured to engage and lock the wire 66 within the slot 63 to prevent removal of the mating end 66a of the wire 66 from the slot 63 while still allowing free sliding movement. An actuator, such as a pusher rod or other device, can be coupled to the mating ends 66a of the wires 66 to slide the wires 66 within the slots 63, thereby selectively extending and retracting the wires 63 from the conduit 62. When the wires 66 are fully extended from the conduit 62, the curved mating ends 66a can also lock against the end of the slot 63 to prevent further deployment of the wires 66, as shown in FIG. 6C. The wires 66 can also vary in length, shape, and configuration. In the illustrated embodiment, the wires 66 differ in length and each wire 66 has a generally elongate linear configuration. The terminal ends 66b of the wires 66 can also optionally be curved or otherwise shaped to prevent tissue trauma following deployment.

In another embodiment, shown in FIGS. 7A and 7B, rather than having the wires can extend in an opposite direction, i.e., toward the mid-portion of the conduit rather than away from the terminal end of the conduit. In particular, FIG. 7A illustrates a graft 70 having a conduit 72 with several wires 74 slidably coupled thereto. Each wire 74 has a mating end 74a that is slidably disposed within a slot 73 formed in the conduit, and a terminal end 74b that extends radially outward from the conduit 72. FIG. 7B illustrates the wires 74 in the initial compressed position. While not shown, the terminal ends 74b of the wires 74 are disposed within the conduit 72 and are located toward the mid-portion of the conduit 72 to prevent the wires 74 from extending through the slots 73. As discussed above, the wires 74 can be actuated by pushing the mating ends 74a of the wires 74 toward the terminal end 72b of the conduit 72, thereby allowing the terminal ends 74b of the wires 74 to extend outward through the slots 73.

In another embodiment, shown in FIGS. 8A and 8B, the graft 80 includes an expandable anchor having wires 84 that are mated to a retention ring 86 that is slidably disposed around the conduit 82. The terminal end 82b of the conduit 82 has a tapered or cone-shaped configuration such that advancement of the retention ring 86 over the terminal end 82b will allow the forward-most end of the retention ring 86 to collapse inward around the conduit 82 causing the trailing end of the retention ring 86 to extend radially outward from the conduit 82. The mating end 84a of the wires 84 can be coupled to the trailing end of the retention ring 86 such that the terminal ends 84b of the wires 84 will extend radially outward from the conduit 82 with the trailing end of the retention ring 86, thereby allowing the expandable member to anchor to tissue. To withdraw the anchor, the retention ring 86 can be pulled back onto the cylindrical section of the conduit 82 causing the ring 86 and the wires 84 attached thereto to swing down onto the conduit 82.

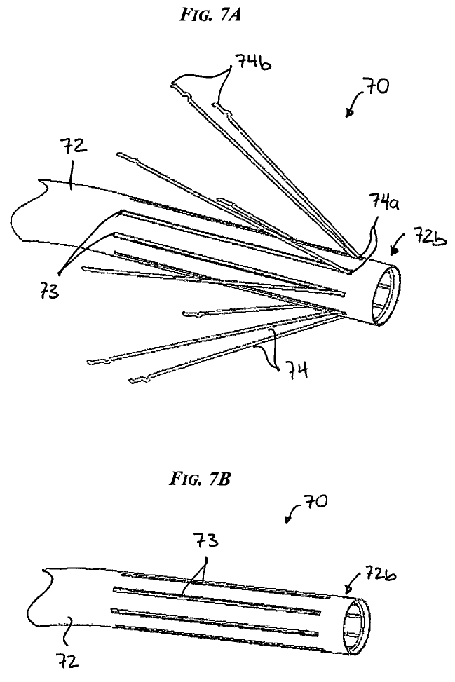

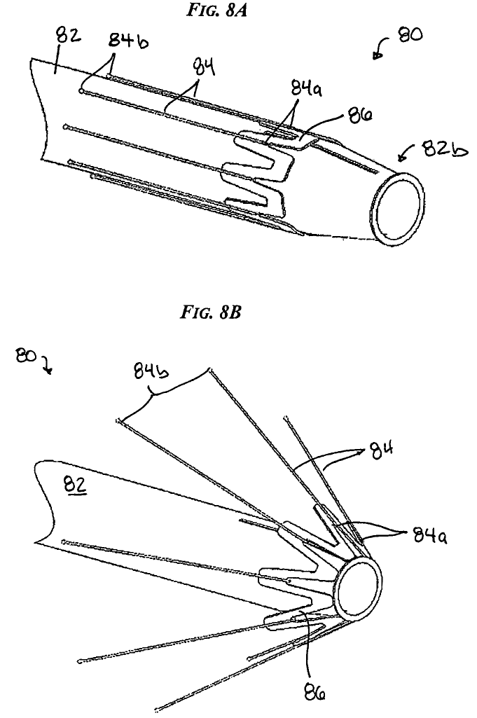

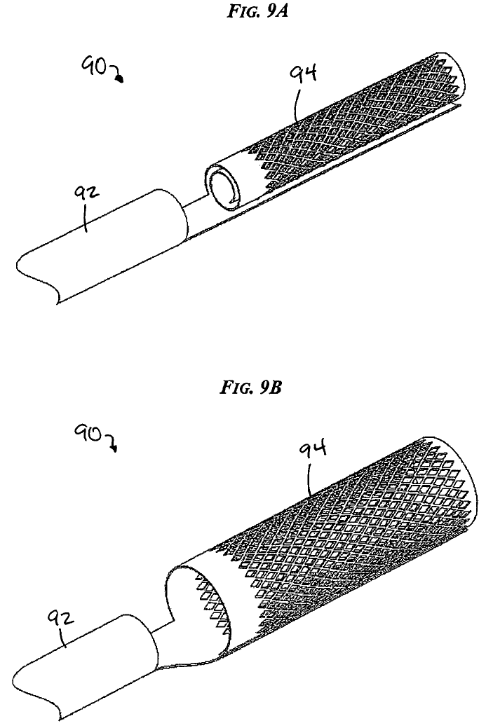

FIGS. 9A and 9B illustrate yet another embodiment of a graft 90 having a conduit 92 with an expandable anchor 94 formed on a terminal end thereof. In this embodiment, the anchor 94 is in the form of a coil having a longitudinal axis that is aligned with a longitudinal axis of the conduit 92. The coil can be wound tightly to allow the coil to be maintained in the conduit 92 prior to deployment. FIG. 9A illustrates the coil in the compressed position, showing the conduit 92 removed for illustrative purposes. The coil can be biased to an expanded position such that advancement of the coil from the conduit 92 will allow the coil to increase in diameter, as shown in FIG. 9B, and thereby expand to engage tissue. The coil can have a variety of configurations, and it can be formed from, for example, a sheet of material that is rolled up. The coil can also include various other features, such as perforations formed therein as shown.

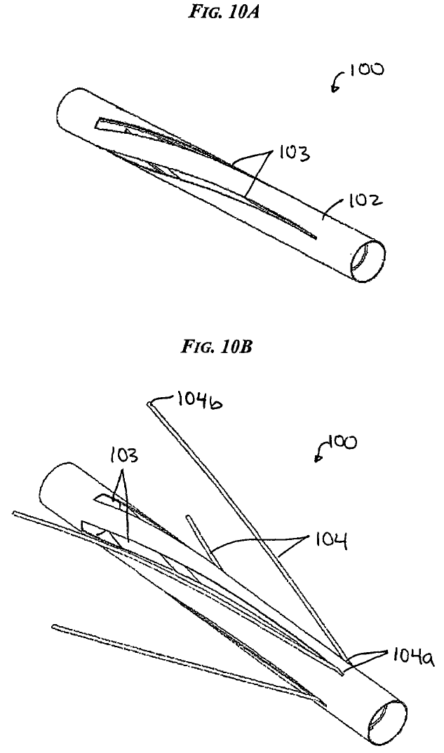

FIGS. 10A and 10B illustrate yet another embodiment of an expandable anchor 100. The anchor 100 is similar to the anchor 64 shown in FIGS. 6A and 6B, however in this embodiment the conduit 102 includes slots 103 that are oriented radially around the conduit 102. A retention ring or other device (not shown) can optionally be attached to a mating end 104a of each wire 104. In use, the wires 104 can be deployed by rotating the retention ring relative to the conduit 102, thereby aligning the wires 104 with the slots 103 formed in the conduit 102. The terminal ends 104b of the wires 104 can thus exit from the slots 103 in the conduit 102 and deploy outward. To withdraw the anchor, the ring can be rotated in the opposite direction causing the wires 104 to retract into the conduit 102.

FIGS. 11A and 11B illustrate yet another embodiment of an expandable anchor 110. In this embodiment, the anchor 110 includes several wire loops 102 which are anchored at one end to a ring 104 which can be removably or fixedly mated to the conduit, or which can form part of the conduit. Prior to deployment, the wire loops 102 can be compressed within the conduit or a delivery sheath. Following delivery of the conduit to its anchor site, the conduit or sheath can be withdrawn causing the wire loops 102 to deploy outward and anchor the conduit in position. To withdraw the conduit, the conduit or delivery sheath can again be slid back over the wire loops 104 to deform the wire loops 104 thereby allowing the conduit to be withdrawn from the body.

In another embodiment, shown in FIGS. 12A and 12B, the expandable anchor 120 can be formed from several expandable strips 124 that can be contained within the conduit 122 or a sheath during delivery, and that can be deployed from the conduit 122 or sheath to anchor the conduit 122 within tissue. The strips 124 can be formed by, for example, cutting several longitudinally-oriented slots in a tubular member and deforming the strips radially outward, as shown in FIG. 12A. As further shown, the terminal ends 124b of the strips can be curved inward to prevent trauma to the tissue following deployment.

FIG. 13 illustrates yet another embodiment of an expandable anchor 130. In this embodiment, the anchor 130 is formed from several hook-shaped wires 134. A mating end 134a of each wire 134 is mated to the terminal end 132b of the conduit 132, or to a ring that mates to the conduit 132, and the terminal end 134b of each wire 134 is curved or hook-shaped to prevent damage to tissue. In an exemplary embodiment, the wires 134 are spaced radially around the conduit 132, and the hook-shaped terminal ends 134b curve inward toward one another. During deliver of the device, the wires 134 can be retained within the conduit or a sheath, and once in position the conduit or sheath can be removed allowing the wires 134 to expand radially outward to anchor the conduit 132 within tissue.

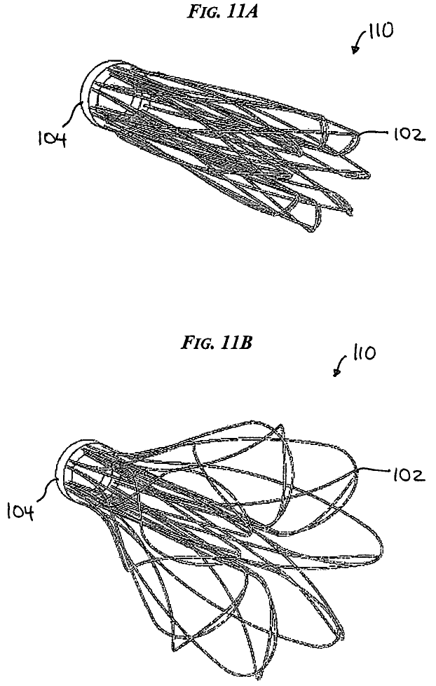

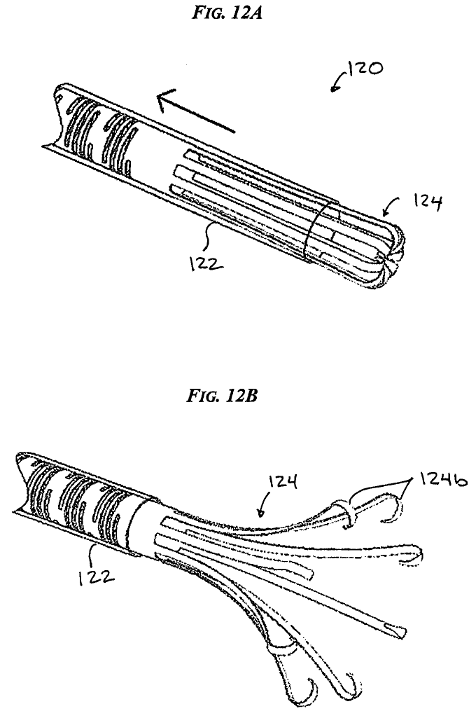

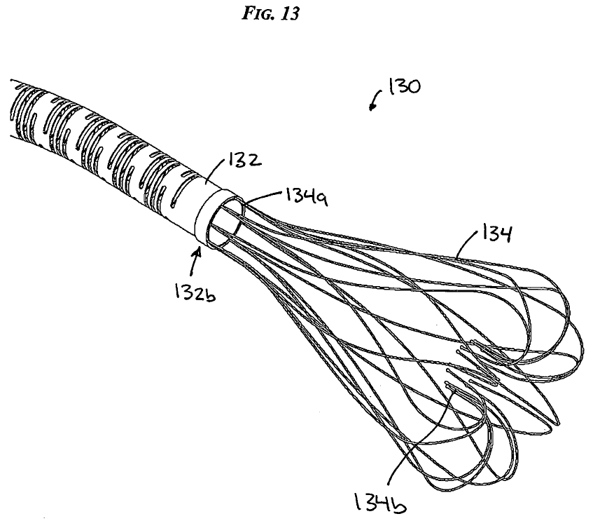

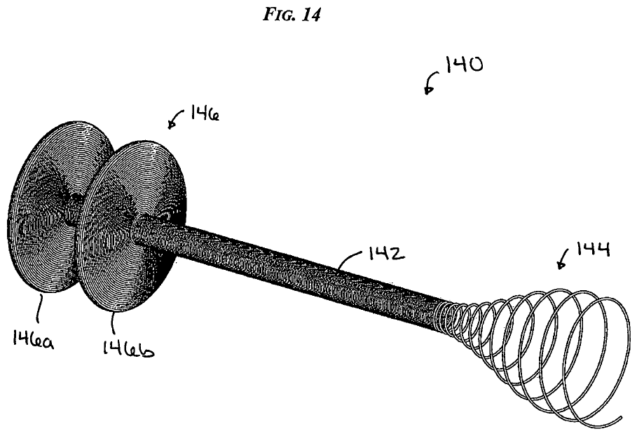

The conduit itself can also have a variety of other configurations. In each of the aforementioned embodiment, the conduit has a generally elongate hollow tubular configuration with several slits or perforations formed therein for allowing blood flow therethrough. FIG. 14 illustrates another embodiment of a graft 140 having a conduit 142 that is formed from a coiled wire. The same coiled wire is further shown as forming a first expandable anchor 144 on one end of the conduit 142, and a second expandable anchor 146 on an opposite end of the conduit. The second expandable anchor 146 includes first and second expandable portions 146a, 146b, each or which is also formed from the same continuous wire used to form the entire graft 140. While the wire is shown having a coiled configuration, the wire can be braided, woven, or otherwise shaped to have the desired shape. The use of a coil to form the conduit 142 is particularly advantageous in that it provides perforations along the length of the conduit 142, thereby helping to maintain a desired pressure within the conduit 142. For example, the coils of the conduit 142 may be in contact with each other in a resting position, and an increase in pressure within the conduit 142 can cause the coils to separate and release blood thereby preventing the pressure from increasing above a certain level. Alternatively the coils may be spaced apart such that blood will continuously leak through the wall and prevent pressure increasing above a certain level within the conduit 142. These advantages also apply when a braiding method is used to construct the graft.

A person skilled in the art will appreciate that the expandable anchors can have a variety of other configurations, and that a variety of techniques, in addition to those previously described, can be used to deploy the anchors. For example, in one embodiment the anchor can be self-expanding. A conduit, sheath, or other retaining element can be disposed around an expandable anchor to compress the anchor. Removal of the conduit, sheath, or other retaining element, i.e., by retracting the conduit, sheath, or other retaining element or by advancing the anchor from the conduit, sheath, or other retaining element, can allow the expandable anchor to self-expand to engage tissue. In another embodiment, an actuator can be used to move the anchor between the compressed and expanded configurations. The actuator can be, for example, a balloon that is disposed within the expandable anchor and that, when inflated, deforms the anchor outward. Another exemplary actuator is a shaft that couples to a portion of the anchor to move the anchor relative to the conduit, thereby compressing and expanding the anchor. In other embodiments, the anchor can be inflated using fluid and/or air.

As previously indicated, the present invention also provides exemplary methods for applying retrograde perfusion of blood at various locations within the body. In an exemplary embodiment, one or more conduits and one or more expandable anchors are used to apply long-term retrograde perfusion of the myocardium, the neurosystem, or a periphery, such as the arm or leg, thereby treating various medical conditions, such as coronary artery disease, stroke, renal failure, etc. The total device, which can be formed from multiple conduit(s) and anchor(s) or from a single member, is collectively referred to herein as a graft. A person skilled in the art will appreciate that the particular configuration of the graft can vary, and that any of the various exemplary conduits and/or expandable anchors can be used in any combination with one another to obtain the desired result.

FIGS. 15-18 illustrate various exemplary grafts having a first end implanted in the left ventricle or left atrium of a heart and a second end implanted in the coronary sinus of the heart for perfusing blood from the left ventricle into the coronary sinus. In FIG. 15, various regions of the heart are labeled as follows: right atrium RA, right ventricle RV, left atrium LA, left ventricle LV, coronary sinus CS, interventricular septum IV, mitral valve MV, and tricuspid valve TV.

In the embodiment shown in FIG. 15, the graft 150 is formed from a conduit having first and second conduit portions 152, 154 that are removably matable to one another. The first conduit portion 152 includes a terminal end 152b that extends through an opening formed between the left ventricle and the right ventricle, i.e., the interventricular septum, and that is anchored to the interventricular septum using an expandable anchor 153 configured as previously described with respect to FIGS. 5A and 5B. The first conduit portion 152 also includes a mating end 152a that is located in the right ventricle and that mated to a mating end 154a of the second conduit portion 154. In particular, the mating ends 152a, 154a of the two conduit portion 152, 154 slide into one another to mate the two components. The terminal end 154b of the second conduit portion 154 extends from the right ventricle through the tricuspid valve and into the coronary sinus, whereby the terminal end 154b of the second conduit portion 154 is anchor within the coronary sinus using an expandable anchor 155 having a cone-shaped configuration, similar to that previously described with respect to FIG. 3.

In use, the graft will allow blood to flow therethrough from the left ventricle to the coronary sinus. In particular, during cardiac systole, blood in the left ventricle is pushed through the conduit of the graft (at a flow rate of for example 50 mls/minute) into coronary sinus, and retro-gradely into venous tributaries across the anchoring mechanism located on the second end of the graft. A series of openings or perforations along the length of the conduit 152 can prevent pressure in the conduit 152 from rising above a peak measurement (for example, 50 mmHg), therefore avoiding damage to the coronary veins which are used for retroperfusion of blood into the myocardium. The quantity, size, and locations of the openings can be calculated to limit a peak pressure obtained within the coronary sinus. For example, the perforations can be enlarged to allow more blood to escape either into the right ventricle or right atrium in the event that arterial inflow via native vessels is improved and less retrograde arterialized blood is required. The perforations can also function to continually wash blood clots from the outer surface of conduit by continuously flushing blood through the perforations. In the event that the coronary artery disease worsens and a greater retro-grade perfusion of arterial blood is required, the perforations in the graft may be blocked by placing a covered stent or other occlusive means within or around the graft to inhibit the leakage of blood into the right atrium or right ventricle and therefore providing greater flow into the coronary sinus and venous branches. In other embodiments, the perforations located on the conduit and/or expandable anchors can be used for the placement of other medical devices, such as pace-maker leads, hypothermic cooling catheters, catheters for infusion of super saturated aqueous oxygen, or for other devices or implants to enhance cardiac function.

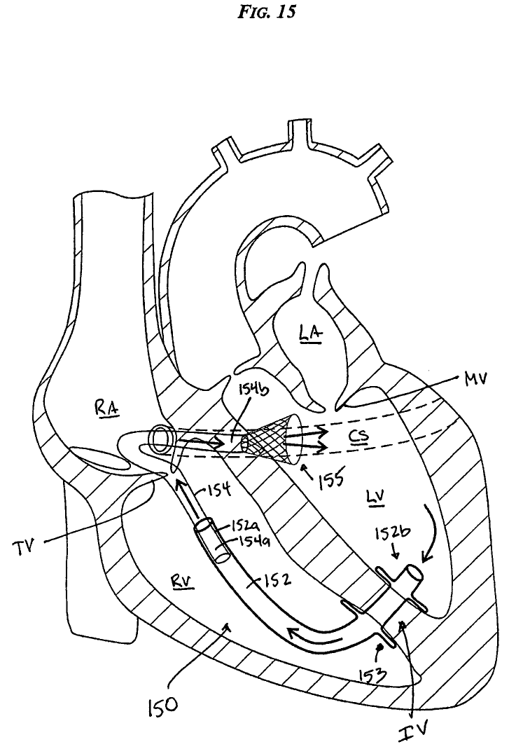

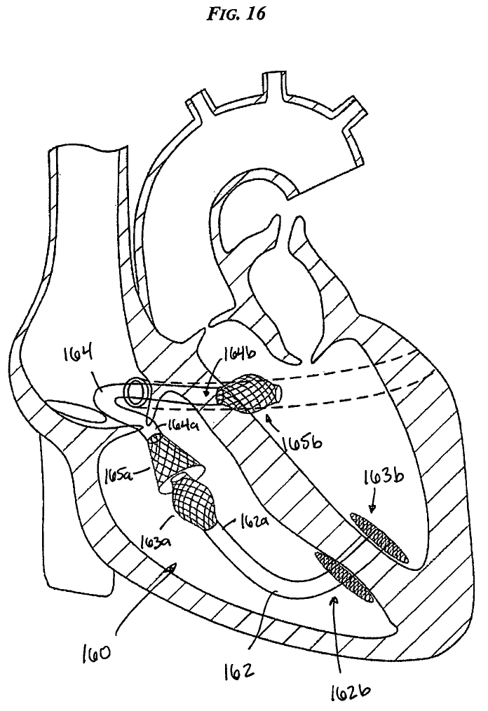

FIG. 16 illustrates another embodiment of a graft 160 having a first conduit portion 162 with a terminal end 162b with an expandable anchor 163b that is anchored within the interventricular septum, and a second conduit portion 164 having a terminal end 164b with an expandable anchor 165b that is anchored within the coronary sinus such that blood can flow from the left ventricle into the coronary sinus. The graft 160 in this embodiment is similar to the graft 150 shown in FIG. 15, however the first and second conduit portions 162, 164 that mate to one another using expandable anchors 163a, 165a, and that also include different expandable anchors 163b, 165b located on the terminal ends 162b, 164b thereof. In particular, the first conduit portion 162 includes an expandable anchor 163b formed on the terminal end 162b thereof that includes first and second expandable portions that are in the form of mesh or wire balloons and that are configured to engage tissue therebetween, and the terminal end 164b of the second conduit portion 164 has an expandable anchor 165b with a generally bulbous oblong shape to facilitate anchoring in the coronary sinus. The first and second conduit portions 162, 164 also include mating ends 162a, 164a having expandable anchors 163a, 165a that are configured to mate to one another. As shown, the mating end 162a of the first conduit portion 162 includes an expandable anchor 163a formed thereon and having a generally bulbous oblong shape such that it is configured to be received within the and to mate to the generally cone-shaped expandable anchor 165a formed on the mating end 164a of the second conduit portion 164. In use, the mating ends of the first and second portions 162, 164 can be positioned within the right ventricle, as shown in FIG. 16, to allow blood to flow through the expandable anchors 163a, 165a and into the right ventricle, thereby decreasing the pressure between the left ventricle and the coronary sinus. In another embodiment, the expandable anchors 163a, 165a on the mating ends 162a, 164a of the first and second conduit portions 162, 164 can be positioned within or adjacent to the opening to the coronary sinus, as shown in FIG. 17.

FIG. 18 illustrates another embodiment of a graft 180 for perfusing blood into the coronary sinus. The graft 180 is similar to the embodiment previously discussed with respect to FIG. 15, however the terminal end 182b of the first conduit portion 182 is implanted in the left atrium. As with the previous embodiments, the first conduit portion 182 can be anchored within the interventricular septum using an expandable anchor 183, such as that previously described with respect to FIGS. 5A and 5B. The first conduit portion 182, or an extension member attached to the first conduit portion 182, can extend from the expandable anchor 183 through the left ventricle, across the mitral valve, and into the left atrium. Blood can thus flow from the left ventricle into the conduit for delivery to the coronary sinus. Positioning of the graft 180 across the mitral valve is particularly advantageous for treating mitral valve regurgitation. Passage of the conduit across valve will result in an inhibition of retrograde flow of blood from the left ventricle into the left atrium and can also help to mechanically inhibit a prolapse of the mitral valve leaflets. In an exemplary embodiment, where the terminal end 182b of the first conduit portion 182 is positioned in the left atrium, the first conduit portion 182, and optionally the second conduit portion 184, can include perforations along most of its length. However, the portion of the conduit located within the mitral valve and the left atrium is preferably free of perforations or openings to prevent blood flow from the left ventricle to the left atrium.

FIG. 18A illustrates one exemplary technique for anchoring the terminal end 182b′ of the first conduit portion 182′ within the left atrium. As shown, a porous disc 183′ is positioned at the terminal end 182b′ of the conduit 182′ so that the mitral valve can close against its surface for improved treatment of mitral valve regurgitation. The terminal end 182b′ can also optionally include a flexible occluder element positioned within the conduit 182′ to prevent blood flow from the left ventricle into the left atrium.

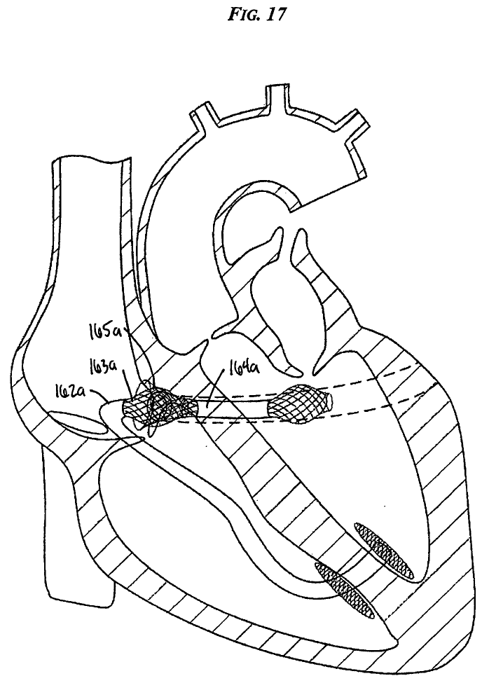

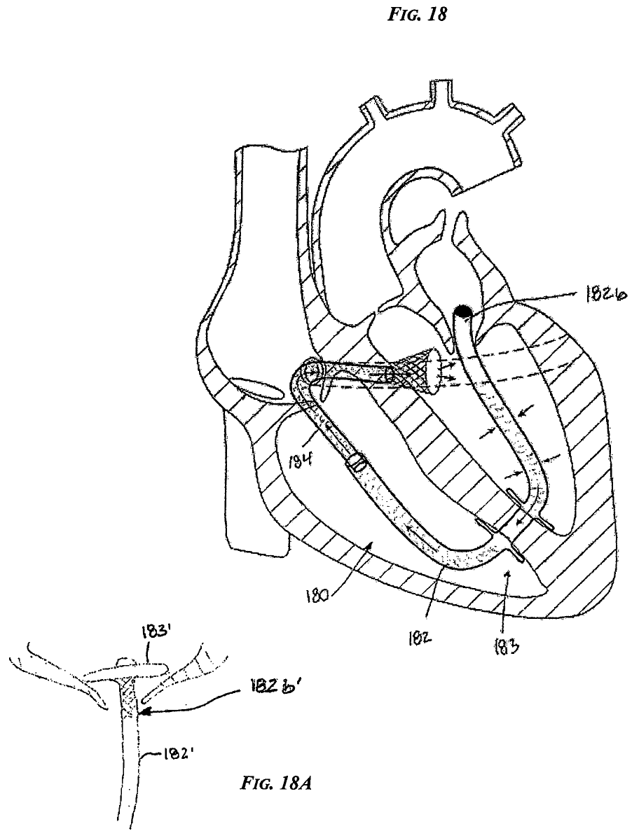

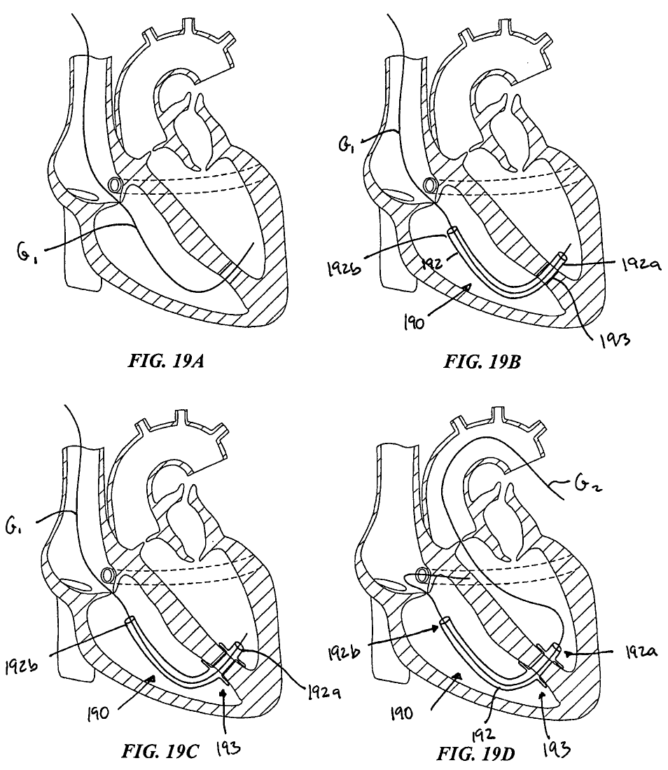

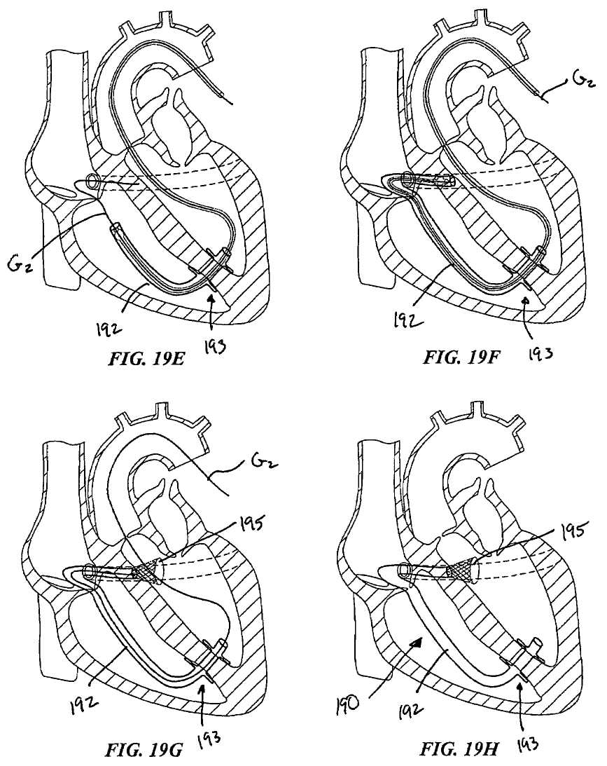

FIGS. 19A-27G illustrate various exemplary techniques for implanting a graft. A person skilled in the art will appreciate that the graft can be delivered either percutaneously or by open surgical techniques. As indicated above, the graft can also optionally be configured to be removed if necessary, or various portions of the graft can optionally be left in-situ and blocked using standard closure devices to close the communication between, for example, the right and left ventricle if so desired.

FIGS. 19A-19H illustrate one exemplary method for creating a venous bypass using a graft 190 having a conduit 192 with a first end 192a that is anchored within the interventricular septum, and a second end 192b that is anchored in the coronary sinus, with the conduit 192 extending from the interventricular septum, through the tricuspid valve, into the right atrium, through the coronary ostium, and into the coronary sinus. The graft 190 may be implanted using a percutaneous translumenal approach by catheterization of the jugular vein. In particular, a cannula is introduced into the jugular vein and is passed into the right atrium, through the triscuspid valve, and into the right ventricle. A puncture is then formed in the interventricular septum using a needle, radio frequency heat, or some other technique for forming a puncture. The puncture hole is then dilated to allow for insertion of the graft therethrough. A guidewire G₁ is then advanced through the cannula to position a second end of the guidewire G₁ within the left ventricle, as shown in FIG. 19A. The first end 192a of the graft 190 is then passed over the guidewire G₁, as shown in FIG. 19B to position an expandable anchor 193 on the conduit 192 within the interventricular septum. Indirect visualization using fluoroscopy, echo-cardiography, or other indirect visualization means can be used to confirm proper positioning of the expandable anchor 193. The anchor 193 is then deployed across the interventricular septum to engage the tissue, as shown in FIG. 19C. The second end 192b of the conduit 192 is then guided into place over a second guidewire G₂ which is introduced through the aorta and advanced into the coronary sinus, as shown in FIGS. 19D and 19E. An expandable anchor 195 disposed within the second end 192b of the conduit 192 can then be deployed to expand the expandable anchor 195, as shown in FIG. 19G, and thereby anchor the second end 192b within the coronary sinus. Exemplary techniques for deploying the expandable anchor were previously discussed herein, and the particular technique used can vary depending on the particular configuration of the anchor. The guidewire G₂ can then be removed via the aortic access (e.g. via femoral artery), leaving the graft 190 in place as shown in FIG. 19H.

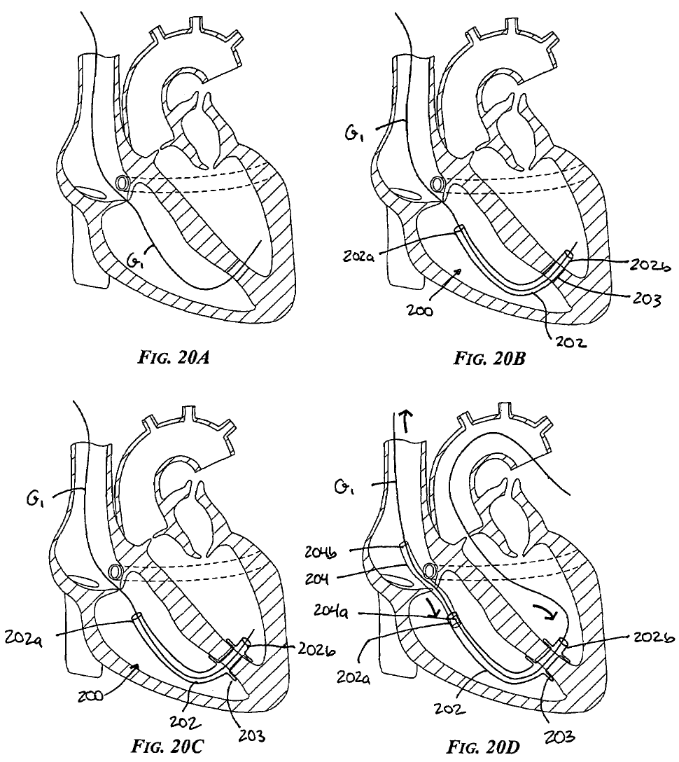

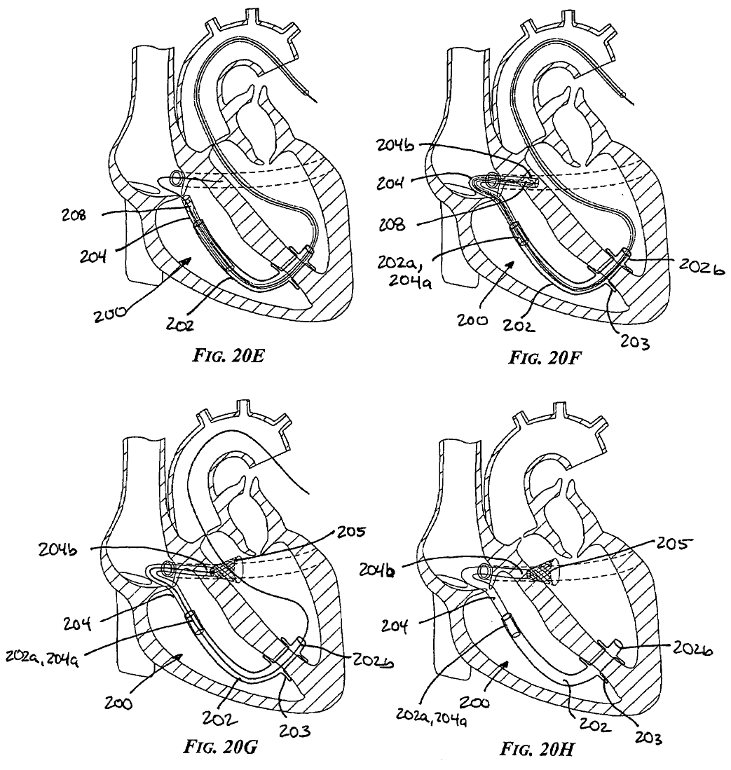

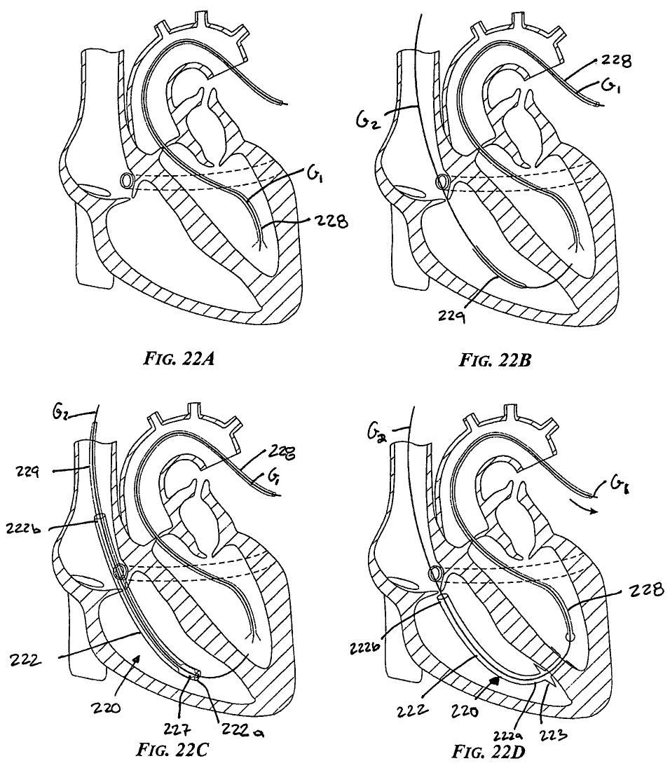

FIGS. 20A-20H illustrate another method for creating a venous bypass. In this embodiment the graft 200 includes a conduit formed from separate first and second conduit portions 202, 204 that mate together. As previously described with respect to FIGS. 19A-19C, a puncture is first formed in the interventricular septum and a guidewire G₁ is positioned to extend through the tricuspid valve, the right ventricle, the interventricular septum, and into the left ventricle, as shown in FIG. 20A. The first conduit portion 202 of the graft 200 is then advanced over the guidewire G₁ to position the terminal end 202b within the left ventricle, as shown in FIG. 20B, and the expandable anchor 203 is then deployed to anchor the terminal end 202a within the interventricular septum, as shown in FIG. 20C. The mating end 202b of the first conduit portion 202 is positioned within the right ventricle. This end 202b may also optionally extend across the tricuspid valve. The second conduit portion 204 of the graft 200 is then advanced over the guidewire G₁ and the mating end 204a of the second conduit portion 204 is inserted into the mating end 202a of the first conduit portion 202 to thereby mate the two portions 202, 204, as shown in FIG. 20D. The first and second conduit portions 202, 204 of the graft 200 can have expandable anchors 203, 205 that are delivered in a preformed state or they can be configured to self-expand after deployment. Following mating of the two portions 202, 204, a second guidewire G₂ is passed through the femoral artery, into the aortic arch, across the aortic valve, and into the left ventricle, as further shown in FIG. 20D. Alternatively, the first guidewire G₁ can be grasped in the left ventricle, using for example a snare, and pulled back via the aorta to exit at the femoral artery and used as described in FIGS. 22C and 22D. The second guidewire G₂ is then passed through the first and second conduit portions 202, 204 and into the coronary sinus, as shown in FIG. 20E. A balloon catheter, or some other attachment mechanism, can then be advanced over the second guidewire G₂ to position a balloon 208 or other anchoring mechanism within the terminal end 204b of the second conduit portion 204. The balloon 208 is then inflated, as shown in FIG. 20E, to engage the terminal end 204b of the second conduit portion 204. The balloon 208, with the second conduit portion 204 of the graft anchored thereto, can thus be advanced along the guidewire G₂ into the coronary sinus, as shown in FIG. 20F. The terminal end 204b of the second conduit portion 204 can be anchored in the coronary sinus using techniques previously described. The guidewire G₂ and balloon catheter can then be removed via the femoral artery, as shown in FIG. 20H.

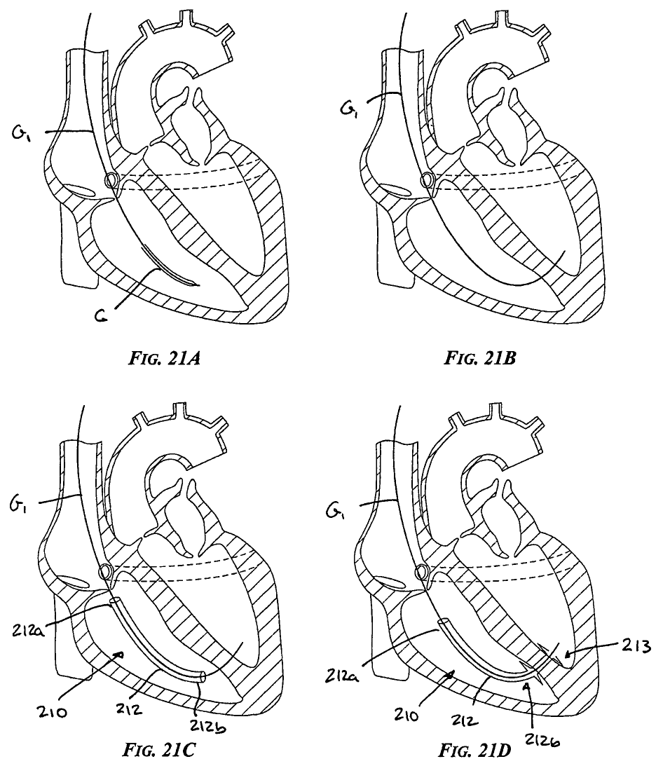

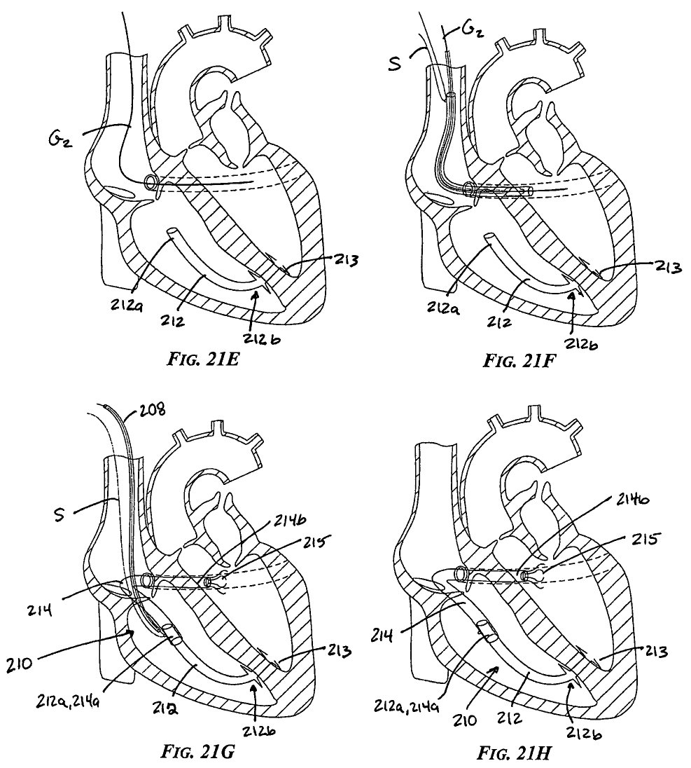

FIGS. 21A-21H illustrate yet another embodiment of a translumenal approach using a method of catheterization of the jugular vein. Following insertion of a cannula C into the jugular vein and through the interventricular septum, as shown in FIGS. 21A and 21B, the terminal end 212b of a first conduit portion 212 of the graft 210 is advanced over the guidewire G₁ down through the tricuspid valve and is deployed across the interventricular septum, as shown in FIGS. 21C and 21D. The expandable anchor 213 on the terminal end 212b of the first conduit portion 212 of the graft 210 is then deployed to anchor the terminal end 212b within the interventricular septum. The mating end 212a of the first conduit portion 212 remains in the right ventricle, or it can extend across the tricuspid valve and into the right atrium. Preparation is now made to deliver the second conduit portion 214 of the graft 210. The mating end 214a of the second conduit portion 214 of the graft 210 may have a suture loop S attached thereto which is of sufficient length to allow the suture to extend through the delivery catheter and out of the patient's body. The mating end 214a of the second conduit portion 214 is delivered through the delivery cannula and advanced along a second guidewire G₂ which has previously been placed into the coronary sinus, as shown in FIGS. 21E and 21F. The terminal end 214b of the second conduit portion 214 can have an expandable anchor 215 formed thereon for anchoring the terminal end 214b within the coronary sinus. The guidewire G₂ and delivery cannula are now removed leaving the mating end 214a of the second conduit portion 214 within the right atrium or the internal jugular vein, as further shown in FIG. 21F. A semi-rigid catheter 208 is then introduced over one loop of the suture loop S exiting the patient, as shown in FIG. 21G. As this semi-rigid catheter 208 is advanced, it will come into contact with the mating end 214b of the second conduit portion 214 of the graft 210. The two components can be held together by pulling the free suture loop S taught. As shown in FIGS. 21G and 21H, the suture loop S can thus be used to steer the mating end 214a of the second conduit portion 214 of the graft 210 to bring it into the right atrium and, depending on its diameter, to mate it with the mating end 212a of the first conduit portion 212 which is located in either the right atrium or the right ventricle. To facilitate this maneuver, a guidewire may be placed through the femoral artery, through the thoracic aorta and retrogradely through the aortic valve and into the left ventricle, passing through the terminal end 212b of the first conduit portion 212 across the interventricular septum and through the first conduit portion 212 to exit from the mating end 212a. The guidewire may then be advanced through the tricuspid valve and into the mating end 214a of the second conduit portion 214 with the help of the suture loop S and the semi-rigid catheter 208, which can be manipulated from outside of the body to facilitate lining up of the mating end 214a of the second conduit portion 214 with the mating end 212a of the first conduit portion 212. Once the second conduit portion 214 has been mated with the first conduit portion 212, the suture loop S can be removed by pulling on one end of the suture. The semi-rigid catheter 208 can also be removed, leaving the graft 210 in place as shown in FIG. 21H.

FIGS. 22A-22H illustrate another variation of a translumenal approach. In this embodiment, a first guidewire G₁ is placed through the femoral artery and is advanced through the thoracic aorta and retrogradely through the aortic valve and into the left ventricle, as shown in FIG. 22A. A grasper or snare 228 is advanced over the first guidewire G₁ and is positioned within the left ventricle, as further shown in FIG. 22A. As previously described with respect to FIGS. 19A-19C, a second guidewire G₂ is inserted through the jugular vein and a cannula 229 is used to puncture through the interventricular septum. A graft 220 is advanced down over the second guidewire G₂ to position an expandable anchor 223 located on the first end 222a of the conduit 222 of the graft 220 within the interventricular septum. The expandable anchor 223 is deployed to engage the interventricular septum, as shown in FIGS. 22B and 22C. The second end 222b of the conduit 222 can remain within the right ventricle. The grasper 228 located in the left ventricle can then be used to grasp the end of the second guidewire G₂ that extends into the left ventricle and to partially withdraw the guidewire G₂ from the patient's body, as shown in FIG. 22D. The trailing end of the second guidewire G₂ can be guided into the coronary sinus, as shown in FIG. 22E. A loop grasper inserted from the jugular end may be used to assist in guiding the trailing end of the guidewire G₂ into the coronary sinus. A balloon catheter 226 or other engagement mechanism can then be advanced over the second guidewire G₂ until the balloon 226a is located within the second end 222b of the conduit 222, as further shown in FIG. 22E. The balloon 226a can be expanded to engage the conduit 222 and to guide the second end 222b of the conduit 222 into the coronary sinus, as shown in FIG. 22F. The second end 222b of the conduit 222 can optionally have an expandable anchor 225 formed thereon for engaging the coronary sinus, as shown in FIG. 22G. The balloon 226a is then deflated and removed, along with the catheter 226 and guidewire G₂, thus leaving the graft 220 in place as shown in FIG. 22H.

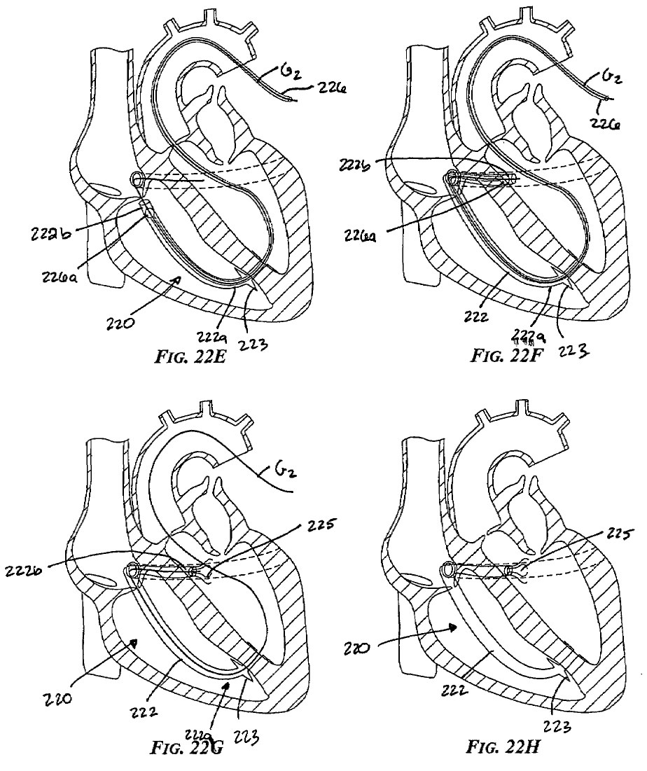

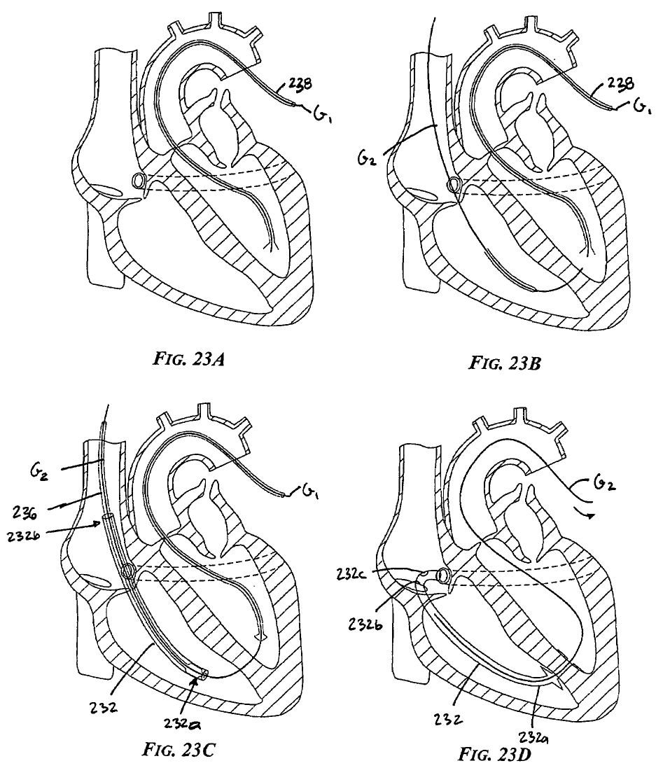

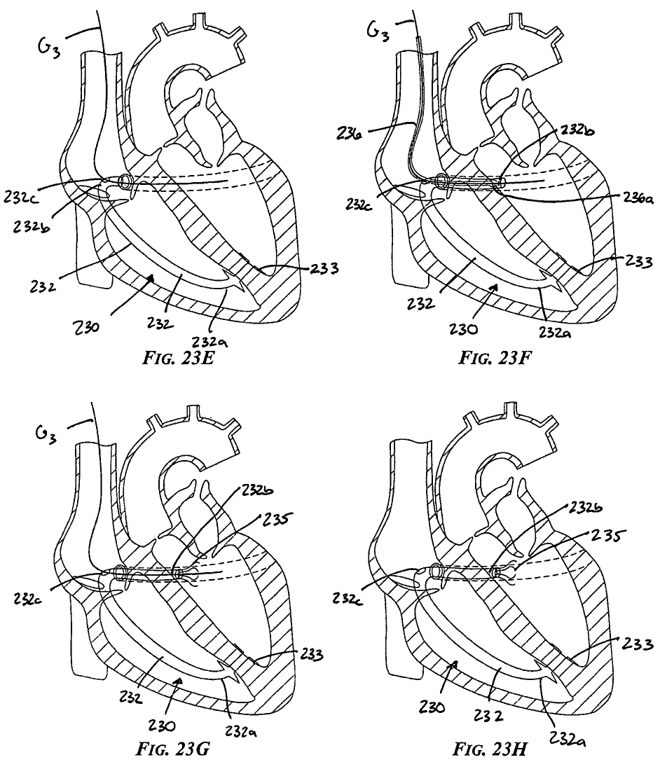

FIGS. 23A-23H illustrate a further variation on a translumenal approach. The method follows the same steps previously described with respect to FIGS. 22A-22D, which are illustrated again in FIGS. 23A-23D. As shown, the first end 232a of the conduit 232 has an expandable anchor 233 for anchoring the first end 232a within the interventricular septum. A balloon catheter 236 can optionally be used to facilitating positioning of the first end 232a within the interventricular septum. The second end 232b of the conduit remains within the right atrium. In this embodiment, when the grasper 238 is used to pull free end of the second guidewire G₂ lying within the left ventricle, the second guidewire G₂ is partially withdrawn from the conduit 232 such that the trailing end of the second guidewire G₂ is no longer located within the second end 232b of the conduit 232 of the graft 230. This will allow the second end 232b of the conduit 232 to return to a pre-formed configuration. For example, as illustrated in FIG. 23D, the second end 232b of the conduit 232 can be biased to a pre-formed curved configuration such that the second end 232b can automatically extend into or towards the coronary sinus. The conduit 232 can optionally be manipulated using a balloon catheter advanced into position over the second guidewire G₂ to facilitate positioning of the second end 232b of the conduit 232 within the entry of the coronary sinus. A third guidewire G₃ is then advanced through the delivery catheter to insert a leading end of the third guidewire G₃ into a port 232c formed in a sidewall of the second end 232b of the conduit 232. The third guidewire G₃ is advanced through the conduit 232 and into the coronary sinus, as shown in FIG. 23E. The balloon catheter 236, or other engagement mechanism, can be advanced over the third guidewire G₃ to position the balloon 236a within the second end 232b of the conduit 232. The balloon 236a can be inflated and used to advance the second end 232b of the conduit 232 into the coronary sinus, as shown in FIG. 23F. The second end 232b of the conduit 232 can optionally have an expandable anchor 235 formed thereon for anchoring the second end 232b within the coronary sinus, as shown in FIG. 23G. The balloon 236b is then deflated and removed, along with the catheter 236 and the guidewire G3, leaving the graft 230 in place as shown in FIG. 23H.

As previously indicated, perforations along the length of the graft can not only facilitate the reduction of pressure within the conduit, improve the flexibility of the conduit, and remove any undesired blood clots which may have formed within or outside the conduit, but they can also be used to allow access to the second end of the graft by placing a guidewire through a perforation and into the conduit, as described above. This guidewire may in turn be placed into a selected coronary vein and a cardiac pacing lead can be placed over the guidewire and delivered to a selected site within the venous vascular tree. Thus, the system allows implantation of a pace-maker lead in addition to retroperfusion of arterialized blood via the venous system.

It should be noted that, in the embodiment shown in FIGS. 23A-23H, the graft can be formed from first and second portions which can be joined together. This can be achieved by placing a guidewire via a femoral or sub-clavian artery into the left ventricle, through the first portion of the graft, and into the second portion of the graft. The mating ends on the first and second portions can then be advanced along the guidewire and into or over one another.

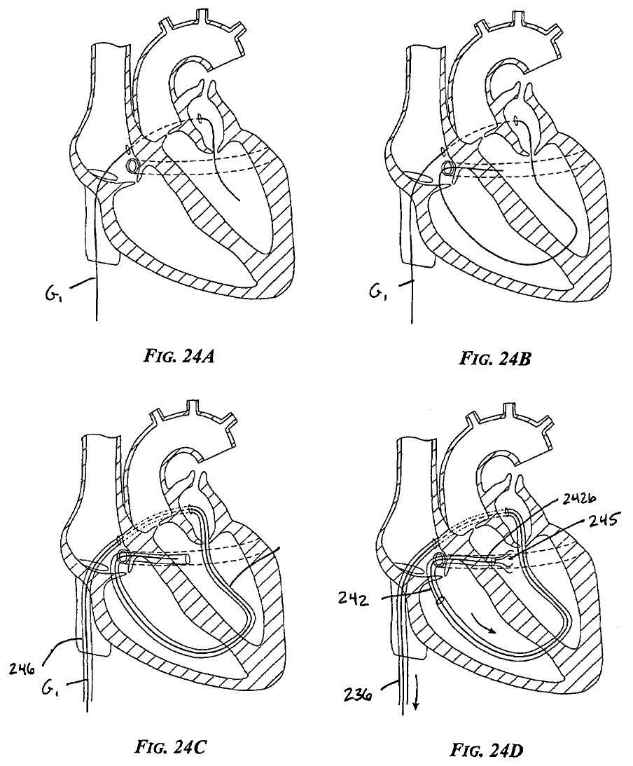

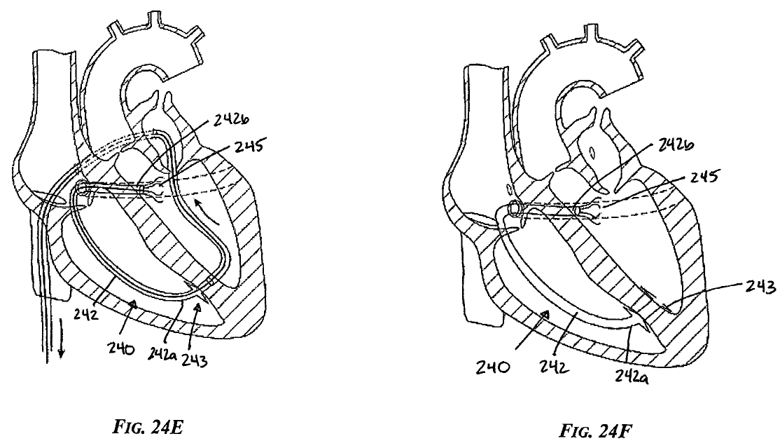

In another embodiment, shown in FIGS. 24A-24F, the graft 240 can be introduced using a trans-septal approach, wherein the graft 240 is introduced through the septum between the right atrium and left atrium. This involves inserting a guidewire G₁ either from the superior vena cava (SVC) or more preferably through the inferior vena cava (IVC), as shown in FIG. 24A. A needle puncture or other puncture techniques can be used to puncture the inter-atrial septum, and the guidewire G₁ can be advanced from the femoral vein through the inferior vena cava and across the inter-atrial septum. The guidewire G₁ is then advanced through the mitral valve and down to the apex of the heart. Under ultrasound control or other indirect visualization techniques, a puncture can be made in the interventricular septum and the guidewire G₁ can be advanced into the right ventricle, through the tricuspid valve and into the coronary sinus, as shown in FIG. 24B. A guide catheter 246 containing the graft 240 can be advanced over the guidewire G₂, through the inter-atrial septum, into the left ventricle, across the interventricular septum, into the right ventricle, across the tricuspid valve, and into the coronary sinus orifice, as shown in FIG. 24C. Once the guide catheter 246 has been placed into the coronary vein, an expandable anchor 245 on the second end 242b of the conduit 242 of the graft 240 can be deployed into the coronary vein, preferably by retracting the guide catheter 246 while holding counter traction on the graft 240 within the lumen of the guide catheter 246. As the guide catheter 246 is withdrawn, the expandable anchor 245 can self-expand to anchor the second end 242b within the coronary sinus, as shown in FIG. 24D. The guide catheter 246 can be further retracted to expose a first portion of an expandable anchor 243 located on the first end 242a of the conduit 242, and the first end 242a of the conduit 242 can be retracted to pull the second anchor 243 against the interventricular septum, as shown in FIG. 24E. Further retraction of the guide catheter 246 will then expose the a second portion of the expandable anchor 243 located on the left ventricular side of the interventricular septum, thereby allowing the two portions of the expandable anchor 243 to engage the interventricular septum therebetween. The guidewire G₁ and catheter 246 can now be fully removed, leaving the graft 240 in place as shown in FIG. 24F. If required, the interventricular portion of the graft can be strengthened by placing an expandable anchor within the portion of the conduit disposed across the interventricular septum. This will inhibit a lapse of the interventricular portion of the graft on systolic contraction of the heart. However, the partially collapsible nature of the interventricular portion will assist in decreasing the pressure within the conduit and decreasing the flow through conduit. This may have a protective effect on veins to which arterialized blood is delivered.

While FIGS. 24A-24F illustrate a graft formed from a single conduit, the graft can alternatively be formed from two or more portions that are matable to one another. In the event that a one piece graft is used, however, it may be necessary for the user to pre-measure the distance between the coronary vein and the interventricular septum in order to select the correct length of the device for deployment. Where a two piece device is used, the graft may be deployed as described above without the need to measure the distance between the coronary vein and the interventricular septum in advance as the conduit will be self adjusting due to the slidability of the two portions of the conduit relative to each other.

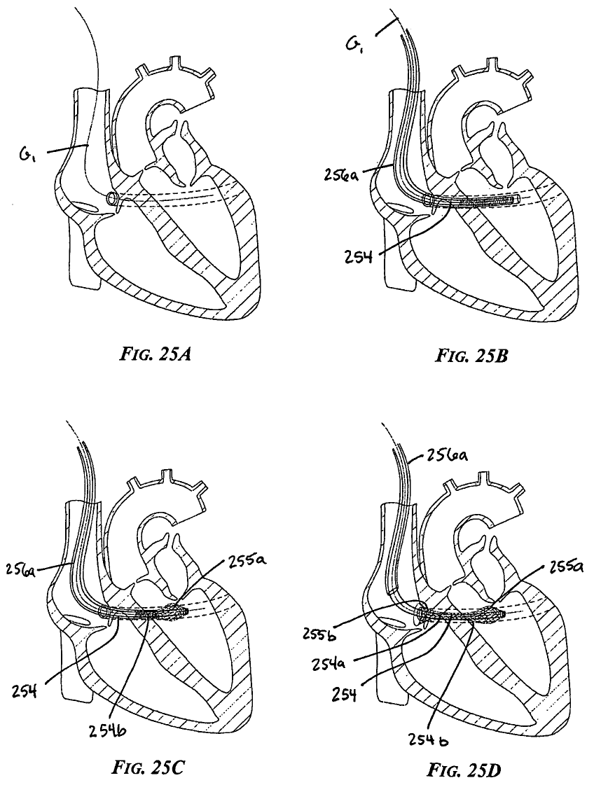

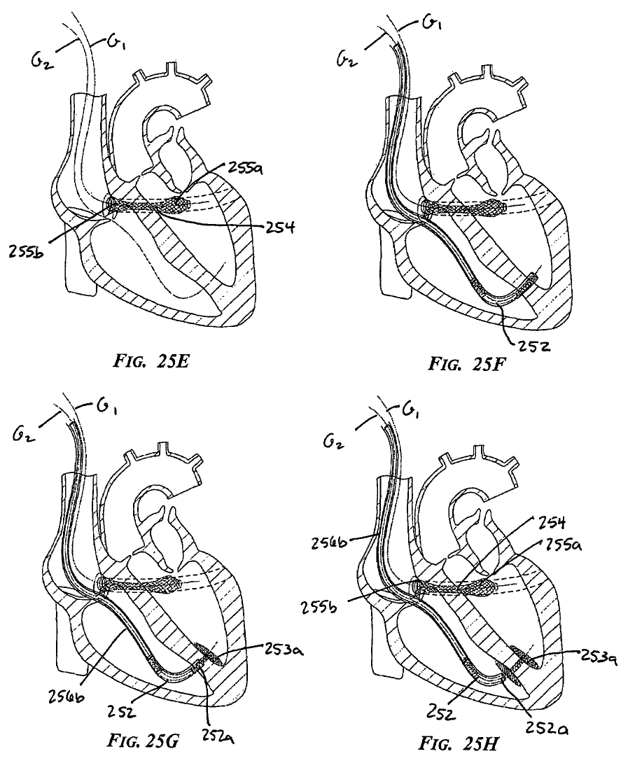

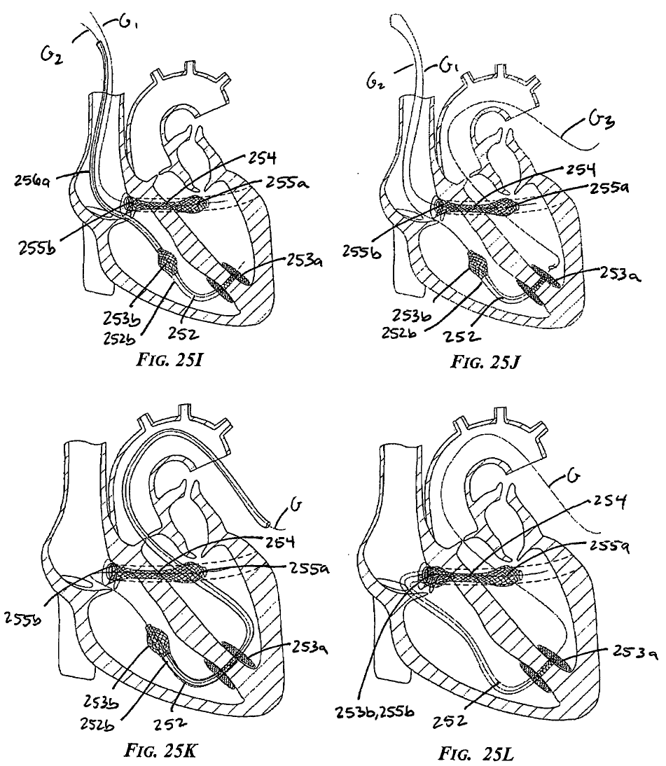

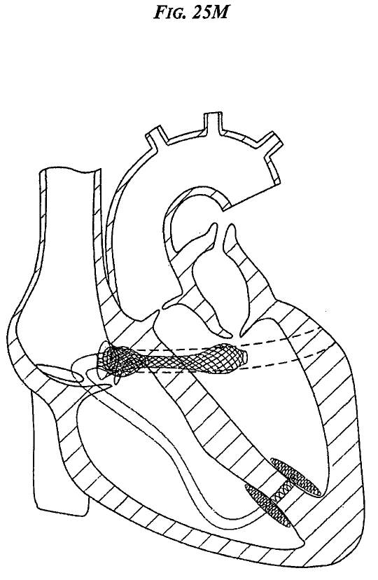

FIGS. 25A-25M illustrate another variation of a translumenal approach. As shown in FIG. 25A, a guidewire G₁ is placed in the jugular vein and passed through the superior vena cava and into the coronary sinus. Following insertion of a cannula into the jugular vein, a second conduit portion 254 of a graft 250 is brought down through the superior vena cava and introduced into the coronary sinus, preferably at a depth of approximately 2-4 cm, as shown in FIG. 25B. An outer delivery catheter 256a disposed over the second conduit portion 254 is then retracted to expose an expandable anchor 255a located on the second end 254b of the second conduit portion 254. As shown in FIG. 25C, the expandable anchor 255a has a bulbous shape with a tubular fixture that binds the self expanding wires of the anchor together. This tubular fixture has a threaded lumen, or other engagement mechanism disposed thereon, which can be attached to a hollow steerable catheter or wire, as shown in FIG. 25C. Once the second end 254b of the second conduit portion 254 is confirmed to be located in the correct anatomical position within the coronary venous system, the hollow steerable catheter can be detached from the engagement mechanism to allow the expandable anchor 255a to expand. An expandable anchor 255b on the first end 254a of the second conduit portion 254 of the graft 250 can then be deployed to engage the opening of the coronary sinus, as shown in FIG. 25D. This can be achieved by retracting the delivery catheter 256a. As shown, the expandable anchor 255b on the first end 254a of the second conduit portion 254 is funnel-shaped and protrudes from the coronary sinus. While not shown, the expandable anchor 255b on the second end 254b of the second conduit portion 254 can optionally be disposed within the right atrium or right ventricle instead of within the opening to the coronary sinus.

Turning to FIG. 25E, a second guidewire G₂ is delivered into the right ventricle and an opening is created between the right ventricle and left ventricle through the interventricular septum using radio frequency or some other technique. Following advancement of the second guidewire G₂ into the left ventricle from the right ventricle, the first conduit portion 252 of the graft 250 is advanced over the second guidewire G₂ and a first portion of an expandable anchor 253a located on the first end 252a of the first conduit portion 252 are deployed within the left ventricle, as shown in FIGS. 25F and 25G. This can be achieved by retracting a delivery catheter 256b disposed over the expandable anchor 253a. As the delivery catheter 256b is further retracted, a wire or other device attached to the first end 252a of the first conduit portion 252 will provide counter-traction to allow a second portion of the expandable anchor 253a to deploy within the right ventricle, as shown in FIG. 25H, or within the right atrium. As the guide catheter 256b is removed, another expandable anchor 253b located on the second end 252b of the first conduit portion 252 will expand, as shown in FIG. 25I.

Several techniques can then be used to mate expandable anchor 253b with expandable anchor 255b. In one embodiment, the expandable anchor 253b located on the second end 252b of the first conduit portion 252 of the graft 250 can be manipulated into the funnel shaped expandable anchor 255b located on the second conduit portion 254 using graspers which may be introduced through the cannula in the internal jugular vein, or using various other techniques such as a suture loop and a guiding cannula. In another embodiment, a third guidewire can be advanced through the femoral artery, into the first conduit portion 252 of the graft 250, through the tricuspid valve, and into the second conduit portion 254 of the graft. A balloon catheter or other device can be advanced over the third guidewire to engage the second end 252b of the first conduit portion 252 of the graft 250. When mated, the balloon catheter can be used to advance the first conduit portion 252 through the tricuspid valve and into the expandable anchor 255b located on the second end 254b of the second conduit portion 254 of the graft 250. In yet another embodiment, the guidewire G₁ in the coronary sinus and the guidewire G₂ placed across the interventricular septum may be joined at the jugular vein insertion site (and pulled back from the femoral artery side to eliminate the loop), to form a continuous wire running from the femoral artery into the coronary sinus, as shown in FIG. 25J. A deployment catheter is advanced over the second guidewire G₂, and the distal end is engaged with the expandable anchor 253b located on the second end 252b of the first conduit 252. The catheter is advanced over the guidewire causing the expandable anchor to engage with the expandable anchor 255b located on the second conduit portion. The deployment catheter is then disengaged and removed along with the guidewire.

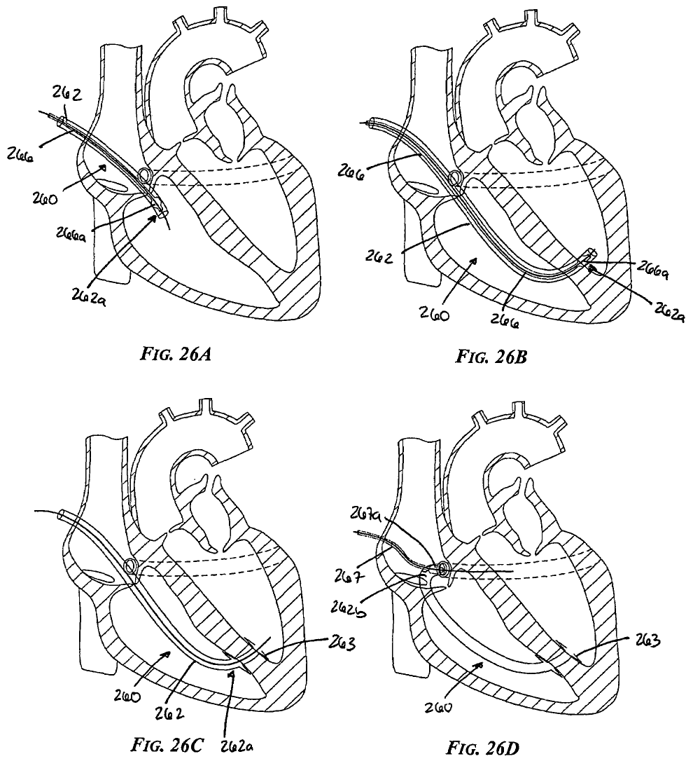

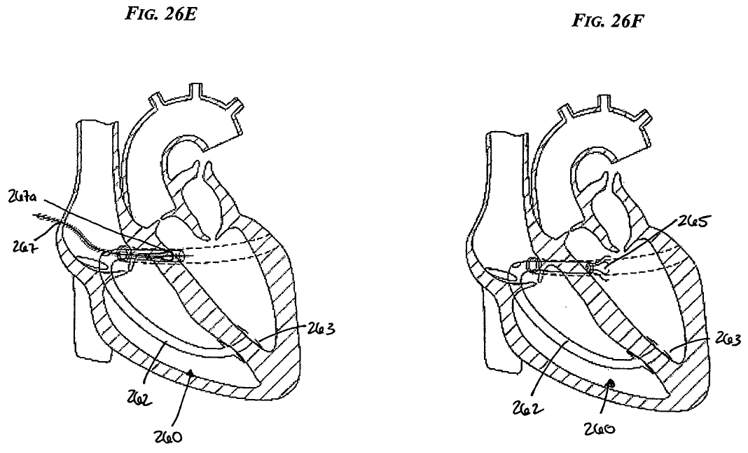

In another embodiment, shown in FIGS. 26A-26F, a graft 260 can be surgically implanted into the heart using a variation of conventional surgical techniques. For example, following a conventional thoracotomy to expose the heart, an incision can be made through the exterior wall of the right atrium. A balloon catheter 266 can be inserted into the conduit 262 and the balloon 266a can be inflated to engage the first end 262a of the conduit 262. The balloon catheter 266 can be used to advance the first end 262a of the conduit 262 through the tricuspid valve and into the right ventricle, as shown in FIG. 26A. As shown in FIG. 26B, the conduit 262 is further advanced across the interventricular septum and into the left ventricle using echo cardiography or other indirect visualization means. The first end 262a of the conduit 262 graft is then anchored to the interventricular septum using an expandable anchor 263, as shown in FIG. 26C. The balloon 266a is then deflated and the catheter 266 is withdrawn. In the event that a balloon is not used, a profiled introducer may be used to insert the first end 262a of the conduit 262 across the interventricular septum. Another balloon catheter 267, or some other attachment mechanism, is then inserted through a side hole formed in the second end 262b of the conduit 262. The balloon 267a is inflated to engage the second end 262b of the conduit 262, and the balloon 267a and conduit 262 are then advanced into the coronary sinus, as shown in FIGS. 26D and 26E. An expandable anchor 265 located on the second end 262b of the conduit 262 can then be deployed to secure the second end 262b within the coronary sinus, as shown in FIG. 26F.

Where the graft is formed from two portions, the first portion may be delivered via the catheter delivery system as described above with the second end being positioned in the right atrium. The second portion of the graft is then placed into the coronary sinus and the first end of the second portion is advanced through the opening in the right atrium. A purse string suture around the opening in the atrium can be used to control blood loss. The second end of the first portion of the graft and the first end of the second portion of the graft are then slidably mated with each other. The loop formed by joining these ends is then slid into the atrium by loosening the purse string suture. The length of the conduit within the heart chambers is then self adjusting and any slack in the conduit is taken up by the slidable nature of the first and second portions of the conduit relative to each other. The purse string in the atrium is pulled tight and the vertical incision in the atrium is repaired.

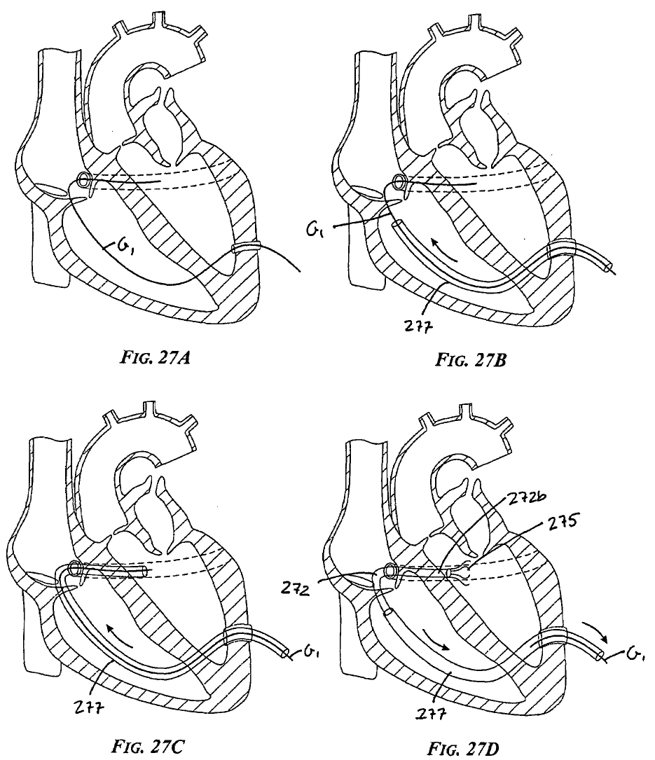

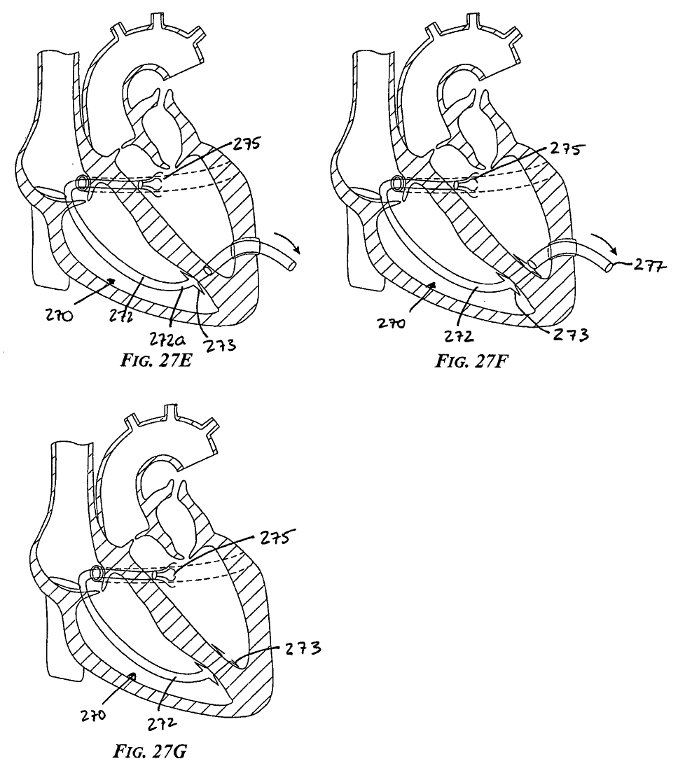

In another embodiment, as shown in FIGS. 27A-27G, a graft 270 may be placed and used as a means of delivering arterialized blood to perfuse the cardiac muscle in the event of an occlusion of either the left or right coronary artery. The surgical approach involves placing a guidewire G₁ in through the apex of the heart, and guiding a graft 270 over the guidewire G₂ into the interventricular septum, retrogradely through the tricuspid valve, and into the coronary vein, as shown in FIGS. 27A-27C. Retraction of a delivery catheter 277 disposed over the graft 270 can allow an expandable anchor 275 on the second end 272b of the conduit 272 of the graft 270 to be deployed within the coronary vein, as shown in FIG. 27D. Further retraction of the deliver catheter 277 will deploy a first portion of an expandable anchor 273 on the first end 272a of the conduit 272 within the right ventricle, as shown in FIG. 27E. With the aid of echo cardiography or other visualization techniques, the first portion of the anchor 273 can be retracted and positioned against the interventricular septum. Further retraction of the catheter 277 can then deploy a second portion of the expandable anchor 273 to cause the portions of the anchor 273 to engage the interventricular septum, as shown in FIG. 27F. The delivery catheter 277 may now be removed from the apex of the heart and the entry site may be sutured or closed by some other surgical technique, as shown in FIG. 27G.

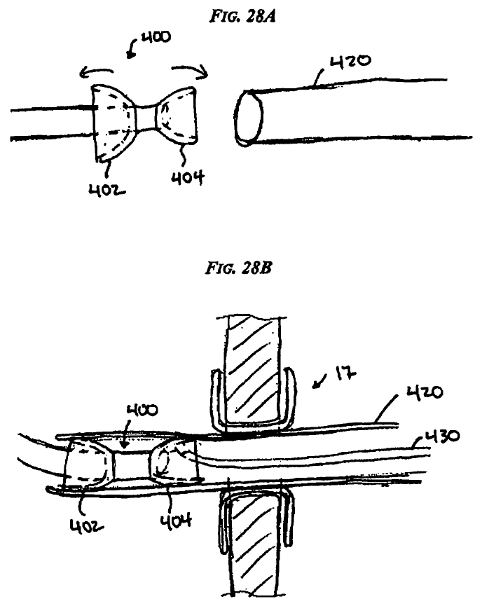

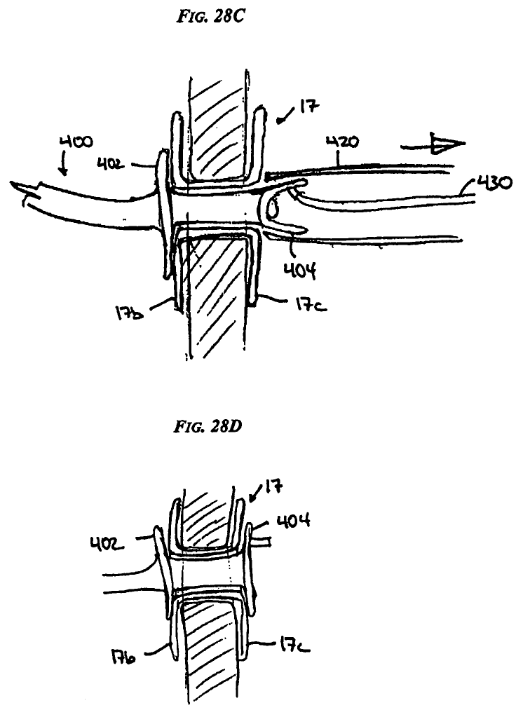

As previously indicated with respect to FIG. 1I, the various methods and devices disclosed herein can also be used in conjunction with a support member that is configured to prevent collapse of tissue disposed therearound. FIGS. 28A-28D illustrate one exemplary method for implanting a graft through the support device 17 of FIG. 1I, which is shown anchored within the interventricular septum. While not shown, the support device can be anchored using various techniques disclosed herein. In an exemplary embodiment, the anchors 17b, 17c on the support device 17 are deformed or flexed to fit within a delivery catheter which is passed through the interventricular septum. The delivery catheter is then retracted to expose the anchor that is positioned on one side of the septum. A delivery wire can be coupled to the attachment member 17d can be used to maintain the support device 17 in position while the delivery catheter is retracted. When the first anchor is expanded, the support member is retracted until the anchor abuts the tissue surface surrounding the septum. The delivery catheter can then be further retracted to expose the second anchor, which will expand to abut the opposed tissue surface. As a result, the tissue will be engaged between the two anchors. Alternatively, the support device 17 can be extended in length causing the anchors 17b and 17c to reduce in diameter. It is then positioned within the delivery catheter 420. The delivery catheter is again retracted to expose one anchor which automatically returns to its original diameter and disc shape. Once this anchor is correctly positioned, the delivery catheter is further withdrawn to expose the second anchor which again expands to its original diameter to abut the opposed tissue surface.

Once the support member is in anchored within the septum, a graft can be anchored through the lumen in the support member and across the septum. The graft can have virtually any configuration, including those disclosed herein. By way of non-limiting example, FIG. 28A illustrates one end of a graft 400 having first and second wing members 402, 404 formed thereon and spaced a distance apart from one another. The graft 400 can be loaded into a delivery device, such as a delivery catheter 420. The wing members 402, 404 can be deformed as shown in FIGS. 28A and 28B such that the wings member 402, 404 are folded inward in opposite directions. This will allow the wing members 402, 404 to move toward and engage tissue or the support device 17 disposed therebetween. With the graft 400 loaded therein, the delivery catheter 420 is advance through the lumen in the support member 17 to position the second end of the graft in the coronary sinus, and to then position the wing members 402, 404 of the first end on opposite sides of the septum. The delivery catheter 420 can then be retracted, as shown in FIG. 28C, to expose one of the wing members, e.g., member 402. FIGS. 28B and 28C illustrate a delivery wire 430 coupled to wing member 404 for maintaining the graft 400 in a fixed position while the delivery catheter 420 is retracted relative thereto. As the catheter 420 is retracted, the wing member 402 will expand and be positioned adjacent to the anchor 17b on the support device 17. Further retraction of the delivery catheter 420 will expose the second wing member 404 to allow the second wing member 404 to expand and be positioned adjacent to the second anchor 17c on the support device 17. The wing members 402, 404 will thus engage the anchors 17b, 17c as well as the tissue therebetween to anchor the graft 400 within the septum, as shown in FIG. 28D.

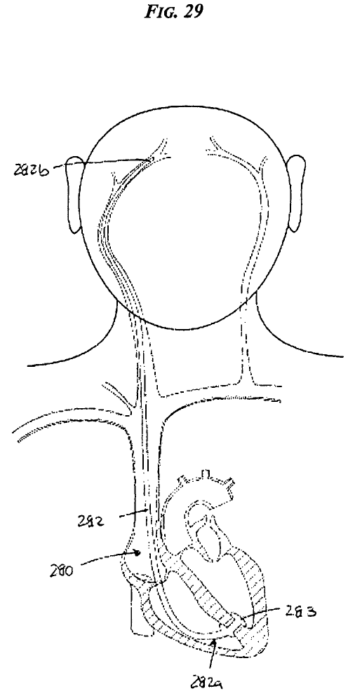

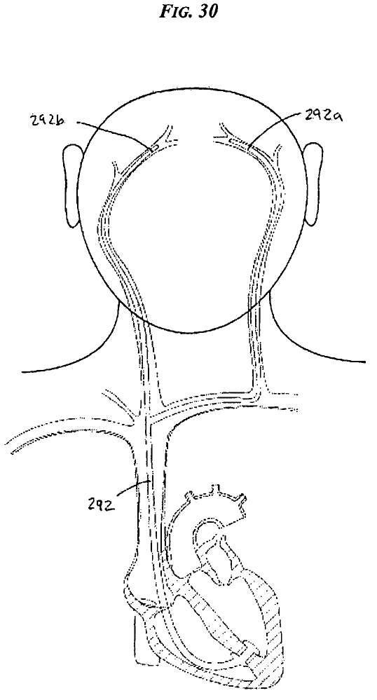

In other embodiments, a graft can be used to overcome the lack of arterial blood reaching a section of brain (stroke) as a result of arterial blockage. This can involve rapidly delivering the patients own arterial blood to the ischemic brain through the cerebral venous system, a system that is redundant, is without valves, and is not effected by athrosclerosis. The technique termed retrograde transvenous neuroperfusion (RTN) is an adoption of coronary retrograde perfusion used for the treatment of acute myocardial ischemia as described below. This RTN technique uses the patients own left ventricle to shunt blood through an innovative conduit across the interventricular septum, through the right ventricle and tricuspid valve into the right atrium, into the superior vena cava, into the internal jugular vein and terminating in the brain, for example in the transverse venous sinus. FIGS. 29 and 30 illustrate two exemplary grafts which can be used for RTN. In the embodiment shown in FIG. 29, the graft 280 includes a conduit 282 having a first end 282a with an expandable anchor 283 formed thereon and a second end 282b that extends into the transverse venous sinus. In the embodiment shown in FIG. 30, the conduit 292 includes a branch portion such that the conduit includes two second ends 292a, 292b. Each end can be positioned within different regions of the brain. As a result arterialized blood is directed retrograde, opposite to normal venous flow, through the central, deep, and superficial sinus veins to reach the capillary bed within the brain. Pressures only moderately above normal venous pressure and well within the acceptable limits are all that is necessary to drive the blood retrogradely towards the ischemic tissue. The blood traverses retrogradely through the capillary bed (bringing oxygen and nutrients to brain tissue) to exit through the redundant venous system.

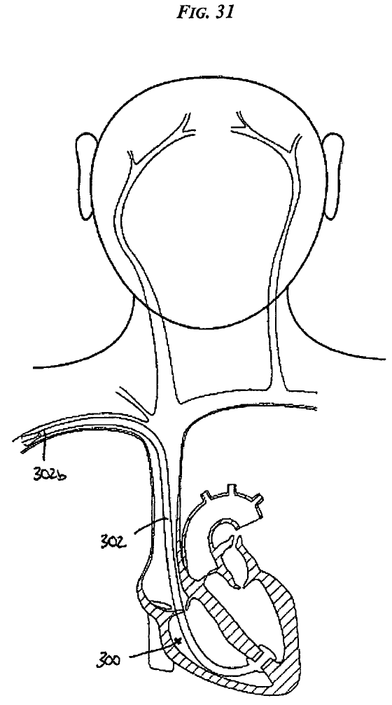

In another embodiment, a graft can be used to create an arteriovenous fistula within the arm or leg region. In one exemplary embodiment, this can be achieved by the retrograde transvenous perfusion of the periphery following placement of a graft that extends from the left ventricle through the interventricular septum, into the right ventricle, retrogradely through the tricuspid valve, into the right atrium, into the superior vena cava, and that terminates secondly in the subclavian vein, as shown in FIG. 31. The graft 300 is similar to the embodiment shown in FIG. 29, however a second end 302b of the conduit 302 is not positioned within the brain but rather is positioned within the subclavian vein. As previously described, the flow rate and pressure at which arterialized blood is delivered into the subclavian vein can be regulated by the configuration of the graft. This device and method of use for retrograde perfusion of a periphery such as the arm results in dilation and maturation of the veins of the arm providing vascular access sites along the extremity. Removal of a patients blood in order to pass it through a dialysis machine and return it at a more first site via another dilated vein on the same limb which has formed as a result of arterialization of the venous system on that limb is now possible as a result of placement of the device creating an arteriovenous fistula.

In the event of eventual failure of vascular access to, for example, a right limb following stenosis of puncture sites or thrombosis, the second end of the conduit may be retracted and removed from right subclavian vein and guided into the left subclavian vein. Such arterialization of the venous system on the left limb will result in further access sites becoming available for hemodialysis as the arteriovenous fistula matures.

It should further be understood that the system may be directed downward into the common femoral vein via the inferior vena cava or more secondly into the right lower limb or left lower limb in order to create vascular access sites if so desired.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A bypass device, comprising:
an implantable hollow flexible conduit configured to be implanted in a human heart, the conduit including first and second ends and a plurality of perforations formed in a sidewall thereof and configured to decrease a pressure of fluid flowing through the conduit; and
at least one expandable anchor formed on the conduit and adapted to expand to engage tissue to anchor at least a portion of the conduit to the tissue, the at least one expandable anchor having a plurality of openings formed therethrough and in communication with the hollow conduit such that blood can flow through the plurality of openings and through the hollow conduit.

2. The device of claim 1, wherein the conduit is formed from a material selected from the group consisting of a metal and a polymer.

3. The device of claim 1, wherein a quantity and a size of the perforations is configured to maintain a maximum pressure within the conduit that corresponds to a maximum pressure obtained within the coronary sinus of a human heart.

4. The device of claim 1, wherein the perforations are formed along a substantial portion of a length of the conduit.

5. The device of claim 1, wherein the at least one expandable member comprises a first expandable anchor formed on the first end of the conduit, and a second expandable anchor formed on the second end of the conduit.

6. The device of claim 5, wherein the first expandable anchor includes first and second expandable portions configured to engage tissue there between.

7. The device of claim 5, wherein the second expandable anchor is formed from a mesh material to allow blood to flow freely therethrough.

8. The device of claim 1, wherein the conduit includes first and second conduit portions that are matable to one another.

9. The device of claim 8, wherein the first conduit portion has the first expandable member formed on a first end thereof, and the second conduit portion has the second expandable member formed on a second end thereof.

10. The device of claim 1, further comprising a one-way valve disposed within at least one of the conduit and the at least one expandable anchor for controlling a direction of blood flow through the device.

11. The device of claim 1, further comprising a cardiac pacing wire disposed through the conduit and at least one of the openings in the at least one expandable anchor.

12. A bypass device, comprising:
a flexible elongate conduit configured to be implanted in a human heart, the conduit including
a lumen extending therethrough and configured to direct blood from a left ventricle, across an interventricular septum, through a right ventricle, and into a coronary sinus of the heart,
a plurality of perforations having a size and a quantity configured to maintain a maximum pressure within the conduit that corresponds to a maximum pressure obtained within the coronary sinus of a human heart, and
at least one expandable anchor configured to engage tissue and anchor at least a portion of the conduit to the tissue, the expandable anchor having a plurality of openings configured to allow blood to flow therethrough.

13. The device of claim 12, wherein the flexible elongate conduit includes first and second conduit portions that are matable to one another.

14. The device of claim 12, wherein the conduit is formed from a material selected from the group consisting of a metal and a polymer.

15. The device of claim 12, wherein the at least one expandable anchor comprises a first expandable anchor formed on a first end of the conduit, and a second expandable anchor formed on a second end of the conduit.

16. The device of claim 15, wherein the first expandable anchor includes first and second expandable portions configured to engage tissue there between.

17. The device of claim 15, wherein the second expandable anchor is formed from a mesh material to allow blood to flow freely therethrough.

18. A method for treating heart disease, comprising:
anchoring a first end of a bypass device within an interventricular septum formed between left and right ventricles of a heart; and
positioning a second end of the bypass device within a coronary ostium of the heart;
wherein the bypass device has a hollow conduit extending from the first end of the device, through the right ventricle, across a tricuspid valve, through the right atrium, into the coronary sinus, to the second end of the device in the coronary ostium such that blood flows from the left ventricle, into the first end, through the conduit, and out the second end into the coronary sinus.

19. The method of claim 18, wherein the conduit includes a plurality of perforations formed therein that decrease a pressure of blood flowing through the conduit.

20. The method of claim 18, wherein positioning the second end of the bypass device within a coronary ostium of the heart further comprises anchoring the second end of the bypass device within the coronary ostium.

21. The method of claim 18, wherein anchoring the first end comprises removing a sheath disposed around an expandable member located on the first end to allow the expandable member to expand to engage tissue.

22. The method of claim 18, wherein anchoring the first end comprises advancing the expandable member from within the conduit to allow the expandable member to expand to engage tissue.

23. The method of claim 18, wherein anchoring the first end comprises inflating a balloon disposed within an expandable member located on the first end to expand the expandable member such that the expandable member engages tissue.

24. The method of claim 18, further comprising positioning a cardiac pacing wire through the bypass device and into tissue in the heart.

25. The method of claim 18, wherein anchoring the first end of the bypass device comprises:
advancing a guidewire through the right atrium and through a puncture formed in the interventricular septum;
advancing the conduit of the device over the guidewire to position the first end of the bypass device within the left ventricle; and
expanding an expandable anchor located on the first end of the bypass device to cause the expandable anchor to engage the interventricular septum, thereby anchoring the first end within the interventricular septum.

26. The method of claim 25, wherein positioning the second end of the bypass device comprises:
inserting a second guidewire through the aorta, into the left ventricle, into the first end and through the conduit of the bypass device, and into the coronary sinus to position a leading end of the second guidewire within the coronary ostium; and
advancing the second end of the bypass device along the guidewire such that the second end of the bypass device is advanced into the coronary ostium.

27. The method of claim 26, wherein advancing the second end of the bypass device along the guidewire comprises:
advancing a catheter over the second guidewire to position an expandable member on the catheter within and adjacent to the second end of the bypass device;
expanding the expandable member on the catheter to engage the second end of the bypass pass; and
advancing the catheter along the guidewire to advance the second end along the guidewire and thereby position the second end in the coronary ostium.

28. The method of claim 26, further comprising expanding an expandable anchor located on the second end of the bypass device to cause the expandable anchor to engage and anchor the second end of the bypass device within the coronary ostium.

29. The method of claim 28, wherein expanding the expandable anchor located on the second end comprises advancing a pusher over the second guidewire and through conduit to push an expandable anchor contained within the second end out of the second end whereby the expandable anchor expands to engage tissue.

30. The method of claim 18, wherein the conduit includes first and second conduit portions that are slidably matable to one another, and wherein positioning the first and second ends of the bypass device comprises:
advancing a first guidewire through the right atrium and through a puncture formed in the interventricular septum;
advancing the first conduit portion of the bypass device over the first guidewire to position the first end of the bypass device within the left ventricle;
expanding at least one expandable anchor located on the first end of the first conduit portion to cause the expandable anchor to engage the interventricular septum, thereby anchoring the first end within the interventricular septum;
advancing the second conduit portion over the first guidewire to slidably mate the second conduit portion to the first conduit portion;
removing the first guidewire;
inserting a second guidewire through the aorta, into the left ventricle, through the first and second conduit portions, and into the coronary sinus to position a leading end of the second guidewire within the coronary ostium; and
advancing the second end of the bypass device along the second guidewire such that the second end of the bypass device is advanced into the coronary ostium.

31. The method of claim 30, wherein expanding at least one expandable anchor located on the first end of the first conduit portion comprises withdrawing a sheath disposed over the first end of the first conduit portion to allow first and second expandable members located on the first end of the first conduit portion to expand and engage the interventricular septum therebetween.

32. The method of claim 30, wherein advancing the second end of the bypass graft along the second guidewire comprises:
advancing a catheter over the second guidewire to position an expandable member on the catheter within and adjacent to the second end of the bypass device;
expanding the expandable member on the catheter to engage the second end of the bypass pass; and
advancing the catheter along the second guidewire to advance the second end along the guidewire and thereby position the second end in the coronary ostium.

33. The method of claim 30, further comprising expanding an expandable anchor located on the second end of the bypass device to cause the expandable anchor to engage and anchor the second end of the bypass device within the coronary ostium.

34. The method of claim 33, wherein expanding the expandable anchor located on the second end comprises advancing a pusher over the second guidewire and through the conduit to push the expandable anchor contained within the second end out of the second end whereby the expandable anchor expands to engage tissue.

35. A method for treating heart disease, comprising:
positioning a hollow elongate conduit within a heart to re-direct blood flow through the conduit from a left ventricle, through an interventricular septum, through the right ventricle, through the right atrium, into the coronary sinus, and into the coronary ostium, the conduit including a plurality of perforations formed therein and configured to maintain a maximum pressure within the conduit that corresponds to a maximum pressure obtained within the coronary sinus of a human heart.

36. The method of claim 35, wherein the hollow elongate conduit includes a first end that is anchor within the interventricular septum, and a second anchor that is positioned in the coronary ostium.

37. The method of claim 36, wherein the second anchor is configured to allow blood to flow therethrough and is configured to at least partially occlude the coronary ostium.

38. The method of claim 36, further comprising removing the device after an extended period of use.
